(19) United States
(12) Patent Application Publication
(10) Pub. No.: US 2023/0285314 A1
(43) Pub. Date: Sep. 14, 2023

(54) DISSOLVABLE MATRICES

(71) Applicant: Thorne HealthTech, Inc., New York, NY (US)

(72) Inventors: Stephen M. PHIPPS, Summerville, SC (US); Michael A. SCHMIDT, Boulder, CO (US); Caleb M. SCHMIDT, Boulder, CO (US)

(21) Appl. No.: 18/179,986

(22) Filed: Mar. 7, 2023

Related U.S. Application Data
(63) Continuation of application No. PCT/US21/49449, filed on Sep. 8, 2021.
(60) Provisional application No. 63/075,803, filed on Sep. 8, 2020.

(51) Int. Cl. A61K 9/70; A61K 36/45; A61K 36/82; A61K 36/185; A61K 36/28; A61K 31/198; A61K 31/4045; A61K 31/352; A61K 31/593; A61K 31/375; A61K 31/4402; A61K 36/22; A61K 47/38; A61K 47/24; A61K 47/36; A61K 47/46; A61K 47/02; A61K 47/10

(52) U.S. Cl. CPC A61K 9/7007; A61K 36/45; A61K 36/82; A61K 36/185; A61K 36/28; A61K 31/198; A61K 31/4045; A61K 31/352; A61K 31/593; A61K 31/375; A61K 31/4402; A61K 36/22; A61K 47/38; A61K 47/24; A61K 47/36; A61K 47/46; A61K 47/02; A61K 47/10

(57) ABSTRACT

Provided herein are dissolvable compositions. Further provided herein are dissolvable compositions comprising prebiotics. Further provided herein are methods of printing dissolvable compositions.

### CROSS REFERENCE

This application is a continuation application of International Patent Application No. PCT/US2021/049449, filed Sep. 8, 2021, which claims the benefit of U.S. Provisional Application No. 63/075,803, filed Sep. 8, 2020, which applications are incorporated herein by reference in their entireties.

### BACKGROUND

Dissolvable compositions comprising pharmaceuticals, nutritional supplements, other substances, and complex mixtures represent an efficient method of delivery or administration. However, these compositions often suffer from low percentages of active ingredients, high hygroscopicity, slow/incomplete dissolution, or structural instability. A need exists for dissolvable compositions which address these challenges.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

The present disclosure relates to solid, dissolvable matrices comprising active agents and excipients that release the active agent when added to a liquid.

Provided herein are pore-containing dissolvable sheets for delivery of one or more active agents into an aqueous medium, the dissolvable sheet comprising: (a) one or more scaffolding agents; (b) one or more blowing agents; and (c) one or more active agents; wherein the sheet, when exposed to an environment at 20 degrees Celsius, 1 atm of pressure, and 45% humidity, absorbs less than 0.003% moisture (w/w) per minute over 235 minutes beginning 5 minutes after exposure to the environment and ending 240 minutes after exposure to the environment. Provided herein are pore-containing dissolvable sheets for delivery of one or more active agents into an aqueous medium, the dissolvable sheet comprising: (a) one or more scaffolding agents; (b) one or more blowing agents; and (c) one or more active agents; wherein the sheet, when placed on a surface of water at a water temperature of 22.2 degrees Celsius, fractures within 2 minutes without mechanical stirring. Provided herein are pore-containing dissolvable sheets for delivery of one or more active agents into an aqueous medium, the dissolvable sheet comprising: (a) one or more scaffolding agents; (b) one or more blowing agents; and (c) one or more active agents; wherein the sheet, when exposed to an environment at 20 degrees Celsius, 1 atm of pressure, and 45% humidity, absorbs less than 0.003% moisture (w/w) per minute over 235 minutes beginning 5 minutes after exposure to the environment and ending 240 minutes after exposure to the environment; and wherein the sheet, when placed on a surface of water at a water temperature of 22.2 degrees Celsius, fractures within 2 minutes without mechanical stirring. Further provided herein are sheets wherein a surface of the dissolvable sheet comprises a plurality of pores having a longest cross-sectional length between 10 and 200 microns, wherein the plurality of pores are present on the surface of the dissolvable sheet at a density of between 10 and 100 pores per square mm. Further provided herein are sheets wherein a surface of the dissolvable sheet comprises a plurality of pores having an average cross-sectional area of 500 to 2000, from 1000 to 5000, or from 1000 to 4000 square microns, wherein the plurality of pores are present on the surface of the dissolvable sheet at a density of between 10 and 100 (or alternatively between 15 and 60) pores per square mm. Further provided herein are sheets wherein the average cross-sectional area of the plurality of pores has a standard deviation less than 7000 square microns, such as between 2000 and 7000 square microns. Further provided herein are sheets wherein the pore-containing dissolvable sheet has a void volume of 3-30%, such as between 4% and 25% or between 5% and 20%. Further provided herein are sheets wherein the sheet, when placed on a surface of water at a water temperature of 22.2 degrees Celsius, fractures within 60 seconds Celsius without mechanical stirring.

Provided herein are pore-containing dissolvable sheets for delivery of one or more active agents into an aqueous medium, the dissolvable sheet comprising: (a) one or more scaffolding agents; (b) one or more blowing agents; and (c) one or more active agents; wherein a surface of the dissolvable sheet comprises a plurality of pores having a longest cross-sectional length between 10 and 200 microns, wherein the plurality of pores are present on the surface of the dissolvable sheet at a density of between 10 and 100 pores per square mm. Further provided herein are sheets wherein a surface of the dissolvable sheet comprises a plurality of pores having an average cross-sectional area of 500 to 2000, from 1000 to 5000, or from 1000 to 4000 square microns, wherein the plurality of pores having the average cross-sectional area of 500 to 2000, from 1000 to 5000, or from 1000 to 4000 square microns are present on the surface of the dissolvable sheet at a density of between 10 and 100 pores per square mm. Further provided herein are sheets wherein the average cross-sectional area has a standard deviation of deviation less than 7000 square microns, such as between 2000 and 7000 square microns. Further provided herein are sheets wherein the pore-containing dissolvable sheet has a void volume of 3-30%, such as between 4% and 25% or between 5% and 20%. Further provided herein are sheets wherein the sheet, when exposed to an environment at 20 degrees Celsius, 1 atm of pressure, and 45% relative humidity, absorbs less than 0.003% moisture (w/w) per minute over 235 minutes beginning 5 minutes after exposure to the environment and ending 240 minutes after exposure to the environment. Further provided herein are sheets wherein the sheet, when mechanically stirred after initial fracture, disintegrates in water within 3 minutes, or within 2.5 minutes, or within 2 minutes. Further provided herein are sheets wherein the sheet, when placed on a surface of water at a water temperature of 22.2 degrees Celsius, fractures within 2 minutes or within 60 second without mechanical stirring. Further provided herein are sheets wherein the one or more scaffolding agents comprises or consists of powdered cellulose. Further provided herein are dissolvable sheets wherein the one or more scaffolding agents comprise or consist of microcrystalline cellulose. Further provided herein are sheets wherein the one or more blowing agents comprise or consist of one or more saponins. Further provided herein are sheets wherein the one or more blowing agents comprise or consist of quillaja extract. Further provided herein are sheets wherein the matrix further comprises an acid/base pair that, when combined, result in the evolution of a gas. Further provided herein are sheets wherein the acid/base pair comprises citric acid. Further provided herein are sheets wherein the acid/base pair comprises a carbonate. Further provided herein are sheets wherein the acid/base pair comprises calcium bicarbonate. Further provided herein are sheets wherein the dissolvable sheet comprises calcium citrate. Further provided herein are sheets wherein the sheet has a maximum thickness of between 50 and 2000 microns, such as between 500 and 1500 microns or between 700 and 1300 microns. Further provided herein are sheets wherein the sheet has an average thickness of between 50 and 2000 microns, such as between 500 and 1500 microns or between 700 and 1300 microns. Further provided herein are sheets wherein the dissolvable sheet has a total surface area of from 0.5 square inches and 20 square inches, from 1 square inch and 10 square inches, or from 2 square inches to 8 square inches when the sheet is treated is having a flat external surface for the purpose determining surface area. Further provided herein are sheets wherein the active agent is or comprises a pharmaceutical composition. Further provided herein are sheets wherein the active agent is or comprises a nutraceutical composition. Further provided herein are sheets wherein the active agent is or comprises a plant extract, animal extract, or fungal extract. Further provided herein are sheets wherein the active agent is or comprises a prebiotic. Further provided herein are sheets wherein the active agent is or comprises a sleep enhancer. Further provided herein are sheets wherein the active agent is or comprises blueberry powder, green tea decaffeinated extract (leaf), pomegranate polyphenol powder, Preforpro (a prebiotic bacteriophage cocktail), Chamomile Extract, L-Theanine, Melatonin, Quercetin Phytosome, Vitamin D3 Veg Powder, Ascorbic Acid 40 Mesh, Zinc Picolinate, mango extract, methyl cobalamin, 1-5-methyltetrahydrofolate, pyridoxal-5-phosphate, or riboflavin-5-phosphate sodium. Further provided herein are sheets wherein the dissolvable sheet comprises at least one hygroscopicity modifier. Further provided herein are sheets wherein the at least one hygroscopicity modifier is or comprises medium-chain triglyceride (MCT) oil powder or carboxymethyl cellulose (CMC) gum. Further provided herein are sheets wherein the dissolvable sheet comprises at least one humectant. Further provided herein are sheets wherein the at least one humectant is or comprises potassium bicarbonate, xylitol, glycerine, or acerola powder. Further provided herein are sheets wherein the sheet comprises no more than 4% water (w/w). Further provided herein are sheets wherein the sheet is substantially devoid of water. Further provided herein are sheets wherein the sheet comprises a water activity of no more than 0.4 after no more than two hours when exposed to 20 degrees C., 1 atm, and 45% relative humidity. Further provided herein are sheets wherein the one or more scaffolding agents are between 0.4% and 40% of the dissolvable sheet by weight. Further provided herein are sheets wherein the one or more blowing agents are between 0.5% and 25% of the dissolvable sheet by weight, such as between 0.5% and 5%, between 3% and 10%, between 7% and 15%, or between 1% and 15%. Further provided herein are sheets wherein the one or more active agents are between 0.05% and 70% of the dissolvable sheet by weight. Further provided herein are sheets wherein the one or more active agents are between 0.05% and 5% of the dissolvable sheet by weight. Further provided herein are sheets wherein the one or more active agents are between 1% and 30% of the dissolvable sheet by weight. Further provided herein are sheets wherein the one or more active agents are between 5% and 30% of the dissolvable sheet by weight. Further provided herein are sheets wherein the one or more active agents are between 1% and 50% of the dissolvable sheet by weight. Further provided herein are sheets wherein the sheet, when placed on a surface of water at a water temperature of 22.2 degrees Celsius, fractures within 50 seconds, within 45 seconds, within 30 seconds, or within 20 seconds without mechanical stirring. Further provided herein are dissolvable sheets wherein the plurality of pores have a cross-sectional area between 1000 and 10,000, between 1000 and 6000, or between 4000 and 10,000 square microns. Further provided herein are dissolvable sheets wherein the plurality of pores are present on the surface of the dissolvable sheet at a density of between 10 and 100, between 10 and 30, or between 20 and 100 pores per square mm. Further provided herein are dissolvable sheets wherein the sheet is a printed sheet. Further provided herein are dissolvable sheets wherein the weight ratio of the one or more scaffolding agents to the one or more active agents is between 1:5 to 3:1, such as between 1:3 and 3:1 or between 1:2 and 2:1.

Provided herein are methods for manufacturing a dissolvable sheet, the method comprising: mixing one or more scaffolding agents, one or more blowing agents, and one or more active agents to from a mixture having a viscosity from 4,000 to 15,000 cP; depositing the mixture onto a surface having a predefined shape; removing water from the deposited mixture to form a solid composition having a water content of less than 4% (w/w). Further provided herein are methods wherein depositing the mixture comprises depositing the mixture onto a stencil. Further provided herein are methods wherein the stencil has a Tillable height of from 0.7 mm to 4.0 mm. Further provided herein are methods wherein the solid composition has a thickness of between 100 microns and 3000 microns, such as between 300 microns and 3000 microns or between 500 microns and 2000 microns. Further provided herein are methods wherein removing the water from the deposited mixture comprises heating the deposited mixture. Further provided herein are methods wherein the deposited mixture is heated by exposure to a temperature of between 45 and 110 degrees Celsius. Further provided herein are methods wherein the deposited mixture is heated for between 15 and 180 minutes. Further provided herein are methods wherein the depositing the mixture onto the surface comprises delivering the material via a printer with a squeegee pressure of 1-100 kgf. Further provided herein are methods wherein the solid composition is a composition according to any composition described herein. Further provided herein are dissolvable sheets formed by any of the methods disclosed herein.

Provided herein are dissolvable matrices comprising: at least 30% (w/w, relative to the dissolvable matrix) of one or more active agents and at least one excipient, wherein the at least one excipient is configured for pore creation and creates structure in the matrix; wherein the dissolvable matrix comprises 1-70% void volume (v/v, relative to the dissolvable matrix), and wherein the matrix is configured to dissolve in an aqueous solution. Further provided herein are dissolvable matrices wherein the at least one excipient is at least one of powdered cellulose and quillaja extract. Further provided herein are dissolvable matrices wherein the dissolvable matrix comprises powdered cellulose and quillaja extract. Further provided herein are dissolvable matrices wherein the powdered cellulose is 10-35% (w/w) relative to the dissolvable matrix. Further provided herein are dissolvable matrices wherein the quillaja extract is 0.5-10% (w/w) relative to the dissolvable matrix. Further provided herein are dissolvable matrices wherein the dissolvable matrix comprises a plurality of pores having an average largest cross-sectional length of about 0.1-100 microns. Further provided herein are dissolvable matrices wherein the dissolvable matrix comprises at least 40% (w/w) of the one or more one active agents relative to the dissolvable matrix. Further provided herein are dissolvable matrices wherein the dissolvable matrix comprises about 50-99% or about 50-90% (w/w) of the one or more active agents relative to the dissolvable matrix. Further provided herein are dissolvable matrices wherein the dissolvable matrix comprises the shape of a cylinder or tablet. Further provided herein are dissolvable matrices wherein the cylinder is no more than 500 microns thick and no more than two inches in diameter. Further provided herein are dissolvable matrices wherein the cylinder is no more than 1000 microns thick and no more than two inches in diameter. Further provided herein are dissolvable matrices wherein the cylinder is 400-500 microns thick and two inches in diameter. Further provided herein are dissolvable matrices wherein the cylinder is 100-500 microns thick and two inches in diameter. Further provided herein are dissolvable matrices wherein the dissolvable matrix has a surface area of at least 8000 mm². Further provided herein are dissolvable matrices wherein the dissolvable matrix comprises a shape of an animal. Further provided herein are dissolvable matrices wherein the dissolvable matrix comprises no more than 8% water (w/w) relative to the dissolvable matrix. Further provided herein are dissolvable matrices wherein the dissolvable matrix comprises no more than 6% water (w/w) relative to the dissolvable matrix. Further provided herein are dissolvable matrices wherein the dissolvable matrix comprises no more than 4% water (w/w) relative to the dissolvable matrix. Further provided herein are dissolvable matrices wherein the aqueous solution is juice, water, tea, milk, coffee, a fermented beverage (beer, wine, kombucha), or soda. Further provided herein are dissolvable matrices wherein the dissolvable matrix is configured to dissolve in water having a temperature of no more than 30° C. in less than 10 seconds. Further provided herein are dissolvable matrices wherein the dissolvable matrix is configured to dissolve in water having a temperature of no more than 30° C. in less than 30 seconds. Further provided herein are dissolvable matrices wherein the dissolvable matrix is configured to dissolve in water having a temperature of no more than 20° C. in less than 60 seconds. Further provided herein are dissolvable matrices wherein the dissolvable matrix is configured to dissolve in water having a temperature of no more than 10° C. in less than 60 seconds. Further provided herein are dissolvable matrices wherein the dissolvable matrix is configured to dissolve in water having a temperature of no more than 5° C. in less than 60 seconds. Further provided herein are dissolvable matrices wherein the dissolvable matrix is configured to dissolve in water having a temperature about 0° C. in less than 60 seconds. Further provided herein are dissolvable matrices wherein the dissolvable matrix is configured to dissolve in water having a temperature about 0° C. in less than 120 seconds. Further provided herein are dissolvable matrices wherein the dissolvable matrix is configured to dissolve in no more than 8 oz of water having a temperature of no more than 20° C. in less than 60 seconds. Further provided herein are dissolvable matrices wherein the dissolvable matrix is configured to dissolve in no more than 8 oz of water having a temperature of no more than 20° C. in less than 30 seconds. Further provided herein are dissolvable matrices wherein the dissolvable matrix is configured to dissolve in an aqueous solution having a pH of 2-10. Further provided herein are dissolvable matrices wherein the void volume is at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, or at least about 70% (v/v) relative to the dissolvable matrix. Further provided herein are dissolvable matrices wherein the void volume is about 1%, about 5%, about 10%, about 15%, about 20%, about 25%, about 30%, about 35%, about 40%, about 45%, about 50%, about 55%, about 60%, about 65%, or about 70% (v/v) relative to the dissolvable matrix. Further provided herein are dissolvable matrices wherein dissolvable matrix comprises a balance of pore size and pore distribution that provides desirable tensile strength, dissolution speed, and moisture transfer rates. Further provided herein are dissolvable matrices wherein the dissolvable matrix is shelf stable.

Provided herein are dissolvable matrices comprising: at least 30% (w/w) of one or more active agents and at least one excipient, wherein the at least one excipient is configured for pore creation and creates structure in the matrix; and wherein the matrix is configured to dissolve in an aqueous solution. Further provided herein are dissolvable matrices wherein the at least one excipient is powdered cellulose or quillaja extract. Further provided herein are dissolvable matrices wherein the at least one excipient comprises powdered cellulose and quillaja extract. Further provided herein are dissolvable matrices wherein the powdered cellulose is 10-35% (w/w). Further provided herein are dissolvable matrices wherein the quillaja extract is 0.5-10% (w/w). Further provided herein are dissolvable matrices wherein the at least one excipient is configured for pore size/distribution modification and/or emulsifier stabilization. Further provided herein are dissolvable matrices wherein the excipient has a D50 of 50-150 microns. Further provided herein are dissolvable matrices wherein the excipient is microcrystalline cellulose. Further provided herein are dissolvable matrices wherein the excipient is tapioca starch, microcrystalline cellulose, or Oat fiber. Further provided herein are dissolvable matrices wherein the excipient is tapioca starch or Oat fiber. Further provided herein are dissolvable matrices wherein the microcrystalline cellulose is 5-15% (w/w). Further provided herein are dissolvable matrices wherein the at least one excipient is an emulsifier. Further provided herein are dissolvable matrices wherein the emulsifier comprises CMC gum. Further provided herein are dissolvable matrices wherein the at least one excipient is a hygroscopicity modifier. Further provided herein are dissolvable matrices wherein the hygroscopicity modifier comprises medium chain triglycerides. Further provided herein are dissolvable matrices wherein the medium chain triglycerides are 1-5% (w/w). Further provided herein are dissolvable matrices wherein the at least one excipient is a mineral ion donor. Further provided herein are dissolvable matrices wherein the mineral ion donor is a calcium salt, e.g., calcium carbonate. Further provided herein are dissolvable matrices wherein the mineral ion donor is 1-10% (w/w). Further provided herein are dissolvable matrices wherein the at least one excipient is a pullulan. Further provided herein are dissolvable matrices wherein the pullulan is 1-5% (w/w). Further provided herein are dissolvable matrices wherein the at least one excipient is a glycerin. Further provided herein are dissolvable matrices wherein the glycerin is 2-15% (w/w). Further provided herein are dissolvable matrices wherein the at least one excipient comprises plant fibers, oils, gums, or collagen. Further provided herein are dissolvable matrices wherein dissolvable matrix comprises a balance of pore size and pore distribution that provides desirable tensile strength, dissolution speed, and moisture transfer rates and is shelf stable.

Provided herein are dissolvable matrices comprising: at least 30% (w/w) of one or more active agents, wherein one or more of the active agents is a prebiotic; and at least one excipient, wherein the matrix is configured to dissolve in an aqueous solution. Further provided herein are dissolvable matrices wherein the prebiotic is a bacteriophage component or a polyphenol component. Further provided herein are dissolvable matrices wherein the matrix comprises at least one bacteriophage component and at least one polyphenol component. Further provided herein are dissolvable matrices wherein the prebiotic is a bacteriophage component. Further provided herein are dissolvable matrices wherein the bacteriophage component comprises one or more lytic bacteriophages. Further provided herein are dissolvable matrices wherein the one or more lytic bacteriophages are of the Siphoviridae or Myoviridae family. Further provided herein are dissolvable matrices wherein the one or more lytic bacteriophages are selected from LH01-Myoviridae, LL5-Siphoviridae, T4D-Myoviridae, or LL12-Myoviridae. Further provided herein are dissolvable matrices wherein the bacteriophage component accelerates the growth of one or more of B. bifidum; B breve; B. animalis subsp. lactis; B. longum; L. acidophilus; L. paracasei; L. plantarum; L. rhamnosus; or B. subtilis. Further provided herein are dissolvable matrices wherein the bacteriophage component supports increases in the concentration of butyrate-producing Eubacteria, decreases the concentration of Clostridium perfringens, or decreases interleukin 4 (IL-4) cytokine. Further provided herein are dissolvable matrices wherein the bacteriophage component is 1-15% (w/w). Further provided herein are dissolvable matrices wherein the bacteriophage component is 1-5% (w/w). Further provided herein are dissolvable matrices wherein the bacteriophage component is about 3% (w/w). Further provided herein are dissolvable matrices wherein the polyphenol component comprises a fruit extract, vegetable extract, or tea leaf extract. Further provided herein are dissolvable matrices wherein the polyphenol component comprises one or more of blueberry extract, green tea extract, and pomegranate extract. Further provided herein are dissolvable matrices wherein the polyphenol component is blueberry extract. Further provided herein are dissolvable matrices wherein the blueberry extract is derived from Vaccinium spp. Further provided herein are dissolvable matrices wherein the Vaccinium spp. is one or more of Vaccinium alaskaense How; Vaccinium ovaliforium Sm; Vaccinium membranaceum L.; Vaccinium uliginosum L.; or Vaccinium cespitosum Mich X Further provided herein are dissolvable matrices wherein the blueberry extract is configured to promote healthy brain and mood, cardiovascular health, blood sugar maintenance, optimal weight, and/or healthy aging. Further provided herein are dissolvable matrices wherein the blueberry extract supports reducing oxidative stress and a healthy response to inflammation in the central nervous system, reduced lipid accumulation in fat cells, and maintenance of blood sugar levels already within a healthy range. Further provided herein are dissolvable matrices wherein the blueberry extract is 10-20% (w/w). Further provided herein are dissolvable matrices wherein the blueberry extract is about 15% (w/w). Further provided herein are dissolvable matrices wherein the polyphenol component is green tea extract. Further provided herein are dissolvable matrices wherein the green tea extract is obtained from Camellia spp. Further provided herein are dissolvable matrices wherein the green tea extract is obtained from Camellia sinensis. Further provided herein are dissolvable matrices wherein the green tea extract comprises at least 19% catechins (w/w). Further provided herein are dissolvable matrices wherein the catechins are selected from (−)-epigallocatechin; (+)-catechin; (−)-epicatechin; (−)-epigallocatechin 3-O-gallate; (+)-gallocatechin 3-O-gallate; (−)-epigallocatechin 3-O-(3′-O-methyl)-gallate; and (−)-epicatechin 3-O-gallate. Further provided herein are dissolvable matrices wherein the green tea extract comprises at least 13% (−)-epigallocatechin 3-O-gallate (EGCG) (w/w). Further provided herein are dissolvable matrices wherein the green tea extract comprises one or more of hydrobenzoic acids; hydroxycinnamic acids; or flavones. Further provided herein are dissolvable matrices wherein the green tea extract further comprises soy phospholipids. Further provided herein are dissolvable matrices wherein the green tea extract supports weight management, cardiovascular health, glucose metabolism, and/or a healthy inflammatory response. Further provided herein are dissolvable matrices wherein the green tea extract is 5-50% (w/w). Further provided herein are dissolvable matrices wherein the green tea extract is 10-20% (w/w). Further provided herein are dissolvable matrices wherein the green tea extract is about 16% (w/w). Further provided herein are dissolvable matrices wherein the polyphenol component is pomegranate extract. Further provided herein are dissolvable matrices wherein the pomegranate extract comprises ellagitannins or punicalagins. Further provided herein are dissolvable matrices wherein the pomegranate extract is obtained from Punica spp. Further provided herein are dissolvable matrices wherein the pomegranate extract is obtained from Punica granatum. Further provided herein are dissolvable matrices wherein the pomegranate extract supports reduction of oxidative damage, cardiovascular health, and/or a healthy immune system. Further provided herein are dissolvable matrices wherein the pomegranate extract is 5-50% (w/w). Further provided herein are dissolvable matrices wherein the pomegranate extract is 5-15% (w/w). Further provided herein are dissolvable matrices wherein the pomegranate extract is 8% (w/w). Further provided herein are dissolvable matrices comprising a hygroscopicity modifier. Further provided herein are dissolvable matrices wherein the hygroscopicity modifier is a medium chain triglyceride (MCT) oil powder. Further provided herein are dissolvable matrices wherein the excipient comprises a pore-creating excipient. Further provided herein are dissolvable matrices wherein the excipient is a pore-creating excipient is a microcrystalline cellulose. Further provided herein are dissolvable matrices wherein the excipient comprises quillaja extract and/or powdered cellulose.

Provided herein are dissolvable matrices comprising: at least 30% (w/w) of one or more active agents, wherein one or more of the active agents is a sleep enhancer; and at least one excipient, wherein the matrix is configured to dissolve in an aqueous solution. Further provided herein are dissolvable matrices wherein the sleep enhancer is selected from chamomile extract, L-theanine, or melatonin. Further provided herein are dissolvable matrices wherein the matrix comprises chamomile extract, L-theanine, and melatonin. Further provided herein are dissolvable matrices wherein the excipient is selected from pullulan, oat fiber, or CMC gum. Further provided herein are dissolvable matrices wherein the excipient comprises quillaja extract and/or powdered cellulose.

Provided herein are dissolvable matrices comprising: at least 30% (w/w) of one or more active agents, wherein one or more of the active agents is an immunity enhancer; and at least one excipient, wherein the matrix is configured to dissolve in an aqueous solution. Further provided herein are dissolvable matrices wherein the active agent is selected from a lecithinized botanical extract, a vitamin, or mineral. Further provided herein are dissolvable matrices wherein the matrix comprises a lecithinized botanical extract, a vitamin, and a mineral. Further provided herein are dissolvable matrices wherein the lecithinized product is quercetin lecithin complex. Further provided herein are dissolvable matrices wherein the vitamin is vitamin D3 or ascorbic acid. Further provided herein are dissolvable matrices wherein the mineral is a zinc salt. Further provided herein are dissolvable matrices wherein the zinc salt comprises a zinc chelate. Further provided herein are dissolvable matrices wherein the excipient is selected from pullulan, tapioca starch, or calcium carbonate. Further provided herein are dissolvable matrices wherein the excipient comprises quillaja extract and/or powdered cellulose. Further provided herein are dissolvable matrices wherein the excipient comprises a pore-creating excipient. Further provided herein are dissolvable matrices wherein the pore-creating excipient is Tapioca starch.

Provided herein are area dissolvable matrices comprising: at least 30% (w/w) of one or more active agents, wherein one or more of the active agents is a performance enhancer; and at least one excipient, wherein the matrix is configured to dissolve in an aqueous solution. Further provided herein are dissolvable matrices wherein the active agent is selected from a mango leaf extract, methylcobalamin, L-methyltetrahydrofolate Ca, and pyridoxal-5-phosphate. Further provided herein are dissolvable matrices wherein the active agent is two or more of mango leaf extract, methylcobalamin, L-methyltetrahydrofolate Ca, and pyridoxal-5-phosphate. Further provided herein are dissolvable matrices wherein the active agent is three or more of mango leaf extract, methylcobalamin, L-methyltetrahydrofolate Ca, and pyridoxal-5-phosphate. Further provided herein are dissolvable matrices wherein the active agent comprises mango leaf extract, methylcobalamin, L-methyltetrahydrofolate Ca, and pyridoxal-5-phosphate. Further provided herein are dissolvable matrices wherein the excipient comprises quillaja extract and/or powdered cellulose. Further provided herein are dissolvable matrices wherein the excipient comprises a pore-creating excipient. Further provided herein are dissolvable matrices wherein the pore-creating excipient is microcrystalline cellulose. Further provided herein are dissolvable matrices wherein the excipient comprises a hygroscopicity modifier. Further provided herein are dissolvable matrices wherein the hygroscopicity modifier is a medium chain triglyceride (MCT) oil powder.

Provided herein are methods for producing a dissolvable matrix comprising: mixing one or more active agents and at least one excipient in a solvent to form a mixture; printing the mixture; and curing the mixture until it comprises no more than 4% water (w/w) to form a dissolvable matrix, wherein the ratio of the one or more active agents to the at least one excipient is at least 50% (w/w). Further provided herein are methods wherein the solvent is water or ethanol. Further provided herein are methods wherein the solvent is at least 30% (w/w) prior to curing. Further provided herein are methods wherein the solvent is 30-60% (w/w) prior to curing. Further provided herein are methods wherein the solvent is 50-70° C. during mixing. Further provided herein are methods wherein the mixture is stirred until the viscosity is 7500-11000 cP. Further provided herein are methods wherein the excipient comprises quillaja extract and/or powdered cellulose. Further provided herein are methods wherein the quillaj a extract is 0.5-2% (w/w). Further provided herein are methods wherein the powdered cellulose is 10-35% (w/w). Further provided herein are methods wherein the method further comprises shaping the mixture using a stencil after step b. Further provided herein are methods wherein the dissolvable matrix comprises a balance of pore size and pore distribution that provides desirable tensile strength, dissolution speed, and moisture transfer rates. Further provided herein are methods wherein the dissolvable matrix is shelf stable.

Provided herein are dissolvable matrices comprising: at least 30% (w/w, relative to the dissolvable matrix) of one or more active agents and at least one excipient, wherein the at least one excipient is configured for pore creation and creates structure in the matrix; wherein the dissolvable matrix comprises one or more pores, and wherein the matrix is configured to dissolve in an aqueous solution. Further provided herein are matrices wherein a cross section of the matrix comprises a pore area of 2-25%. Further provided herein are matrices wherein a cross section of the matrix comprises pores having an average size of 1000-10,000 square microns. Further provided herein are matrices wherein a cross section of the matrix comprises pores having standard deviation of 2000-15,000 square microns. Further provided herein are matrices wherein a cross section of the matrix comprises pores having a size of 200-180,000 square microns. Further provided herein are matrices wherein the matrix comprises pores having an average volume of 25,000-30,000 cubic microns. Further provided herein are matrices wherein a cross section of the matrix comprises pores having standard deviation of 2,000-30,000 square microns. Further provided herein are matrices wherein the matrix comprises pores having a volume of 1000-350,000 cubic microns.

Any aspect or embodiment described herein can be combined with any other aspect or embodiment as disclosed herein.

### BRIEF DESCRIPTION OF THE DRAWINGS

FIG. 1 depicts a workflow for the formulation of dissolvable compositions based on an active ingredient's properties (hygroscopic, lipophilic, and presence of lecithin component).

FIG. 2 depicts an exemplary dissolvable composition produced by the methods described herein.

FIG. 3 depicts an orientation of X axis and Z axis preparations of dissolvable compositions (e.g., disks) for scanning electron microscope (SEM) imaging.

FIG. 4A depicts SEM images used to calculate quantitative characteristics for a dissolvable composition comprising prebiotics. The dissolvable composition was generated using the methods of matrix 1a.

FIG. 4B depicts histograms of pore size distributions for a dissolvable composition comprising prebiotics. The dissolvable composition was generated using the methods of matrix 1a. Note the histograms correspond to the SEM images in FIG. 4A.

FIG. 5A depicts SEM images used to calculate quantitative characteristics for a dissolvable composition comprising ingredients to promote the immune system. The dissolvable composition was generated using the methods of matrix 2a.

FIG. 5B depicts histograms of pore size distributions for a dissolvable composition comprising ingredients to promote the immune system. The dissolvable composition was generated using the methods of matrix 2a. Note the histograms correspond to the SEM images in FIG. 5A.

FIG. 6A depicts SEM images used to calculate quantitative characteristics for a dissolvable composition comprising ingredients to promote performance. The dissolvable composition was generated using the methods of matrix 2.

FIG. 6B depicts histograms of pore size distributions for a dissolvable composition comprising ingredients to promote performance. The dissolvable composition was generated using the methods of matrix 2. Note the histograms correspond to the SEM images in FIG. 6A.

FIG. 7A depicts SEM images used to calculate quantitative characteristics for a dissolvable composition comprising prebiotics. The dissolvable compositions were generated using the methods of matrix 1a. A comparison to conditions without quillaja or threefold excess quillaja is also shown.

FIG. 7B depicts SEM images used to calculate quantitative characteristics for a dissolvable composition comprising ingredients to promote the immune system. The dissolvable compositions were generated using the methods of matrix 2a. A comparison to conditions without quillaja or threefold excess quillaja is also shown.

FIG. 7C depicts SEM images used to calculate quantitative characteristics for a dissolvable composition comprising ingredients to promote performance. The dissolvable compositions were generated using the methods of matrix 2. A comparison to conditions without quillaja or threefold excess quillaja is also shown.

### DETAILED DESCRIPTION

The present disclosure relates to solid, dissolvable matrices comprising active agents and excipients that release the active agent when added to a liquid. The matrices' formulations of the present disclosure take into consideration three dominant aspects that are desired for the finished product to perform against manufacturing forces, environmental forces, and to satisfy the end-user experience. This would then take into consideration the need to control the balance of pore size and pore distribution to ensure tensile strength, dissolution speed, and moisture transfer rates to ensure a finished product that is shelf stable and meets the needs of the end user. Solid, dissolvable matrices of the present invention have a surprisingly advantageous balance of water solubility (which is promoted in part by a higher number and volume of pores) and tensile strength/stability of the matrix (during manufacture, transportation and storage). However, too many or too large of pores reduces the strength of the matrix, and such a matrix may be brittle and/or fracture. Indeed, certain excipients described herein provide an ideal number of pores when present in an matrix when at a certain amount; however, above that certain amount, the pores are undesirably irregular, large, and even form channels that transverse the matrix. It has been challenging to discover dissolvable matrix formulations that provide the proper balance to provide a desirable final product.

Surprisingly, a matrix's formulation, including amounts and types of excipients and other ingredients, will vary depending on characteristics of its active agent. The types and amounts of active agents, excipients, and other ingredients provide the matrix an architecture, comparing particular pore sizes, ranges, distribution, density, and volume, that affects the matrix's dissolution rate and ability to load desirable amounts of active agent in the matrix. As examples, illustrative matrices of the present disclosure include pore forming (blowing and scaffolding, respectively) quillaja and powdered cellulose as excipients; these excipients serve as a blowing agent coupled with a scaffolding agent, respectively, which promote pore formation and help create structure in the matrix. Together, the dissolvable matrices of the present disclosure provide consistent delivery of active agents having various characteristics to a liquid.

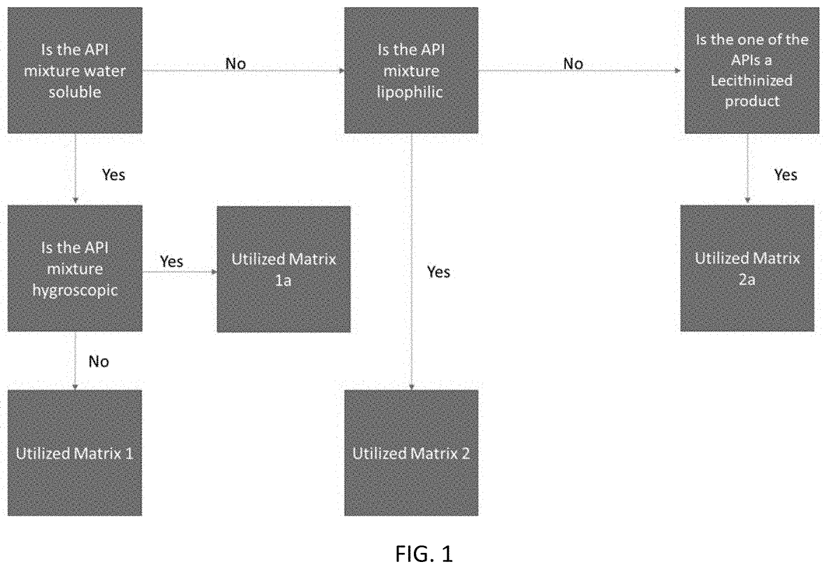

FIG. 1 illustrates methods for designing a matrix's formulation based on characteristics of its active agents. Whether or not the active agent (or combination of more than one active agent) is water soluble is considered first. Agent(s) are deemed water soluble when the material dissolves in a water-based solvent and water insoluble when the material does not dissolve in a water-based solvent. In some instances, solubility in water is determined by a threshold amount of agent/mL solvent at a temperature for a given time. If the active agent(s) is water soluble, the next consideration is whether or not the active agent is hygroscopic, i.e., water absorbing or adsorbing. In some instances, agent(s) are deemed hygroscopic when raw materials that pick up >5% mass at room temperature within nominal relative humidity conditions and not hygroscopic when raw materials that pick less than 5% at room temperature within nominal rh (relative humidity) conditions. In some instances, nominal relative humidity conditions comprise 30-50% relative humidity. In some instances, a hygroscopic agent picks up >5% mass when placed in an environment for one day at standard temperature and pressure in an environment with a relative humidity of 30%. In some instances, a non-hygroscopic agent picks up less than 5% mass at standard temperature and pressure for one day in an environment with a relative humidity of 30%. If the active agent (or combination of more than one active agent) is not hygroscopic, a formulation of dissolvable matrix 1, as disclosed herein, can be used; if the active agent(s) is hygroscopic, a formulation of dissolvable matrix 1a, as disclosed herein, can be used. For active agents (or combinations of more than one active agent) that are water insoluble, the next consideration is whether or not the active agent(s) is lipophilic, i.e., tends to dissolve in fats, oils, lipids, and non-polar solvents. Agent(s) are deemed lipophilic when there is a notable bilayer formation within system and to which the addition of emulsifier rectifies the bilayer formation and not lipophilic when there is no notable bilayer formation within system. In some instances, lipophilicity is defined by a log P value. If the active agent(s) is lipophilic (as determined by a threshold value), a formulation of dissolvable matrix 2, as disclosed herein, can be used. For example, the threshold value for a lipophilic agent is a log P more than 2. If the active agent(s) is not lipophilic and the active agent is lecithin, i.e., a lipid-based vesicular delivery system, a formulation of dissolvable matrix 2a, as disclosed herein, is used.

Certain features of the formulations are described in Table 1:

TABLE 1

| | | | | Matrix 2a: |
|---|---|---|---|---|
| | Matrix 1: | | | Water |
| | Water | Matrix 1a: | | dispersible |
| | soluble, | Water | Matrix 2: | but with a |
| | not | soluble- | Water | lecithinized |
| | hygroscopic | hygroscopic | dispersible | extract |
| Architecture standard | 15-30% | 15-30% | 12-20% | 12-20% |
| (e.g., Powdered | | | | |
| Cellulose) | | | | |
| Architecture addition | N/A | N/A | 1-13% | 1-13% |
| (e.g., Oat fiber or | | | | |
| tapioca starch, | | | | |
| different particle | | | | |
| size/shape to allow | | | | |
| for more structure; | | | | |
| adds more fibrous | | | | |
| material to get | | | | |
| improved | | | | |
| dissolution) | | | | |
| Pore control | 5-10% | 5-10% | N/A | N/A |
| (depth/distribution), | | | | |
| e.g., Microcrystalline | | | | |
| cellulose. Very low | | | | |
| particle size (D50 of | | | | |
| about 100 micron) | | | | |
| Blowing agent | 1-3% | 1-3% | 3.5-8% | 2-7% |
| (e.g., Quillaja, | | | | |
| saponins) | | | | |
| Hygroscopic | N/A | 1-5% | N/A | N/A |
| modification (e.g., | | | | |
| MCT oil; still need to | | | | |
| create more moisture | | | | |
| control, so as not | | | | |
| to disturb the | | | | |
| disintegration; the | | | | |
| higher the amount | | | | |
| of MCT oil, the | | | | |
| more it turns into | | | | |
| a lump) | | | | |
| Mineral Addition | N/A | N/A | N/A | 0.5-20% |
| (for reverse | | | | |
| solubility); it allows | | | | |
| for a secondary | | | | |
| mechanism for the | | | | |
| lecithinized | | | | |
| ingredients for the | | | | |
| blowing agent to | | | | |
| work properly | | | | |

% are expressed as (w/w) of the dissolvable matrix after drying.

The solid dissolvable matrices of the present disclosure comprise one or more active agents. The one or more active agents provide a useful biological activity to a consumer; examples of such active agents include nutritional supplements, vitamins, minerals, drug therapeutics, botanicals, amino acids, proteins, oligopeptides, polypeptides, lipids (including but not limited to fatty acids, phospholipids, ceramides, sphingolipids, etc.), carbohydrates, polysaccharides, probiotics, and prebiotics. Accordingly, the matrices of the present disclosure allow for rapid preparation, by an end user and when needed/desired, of consumable liquid compositions comprising pharmaceuticals, nutritional supplements, and other substances. Notably, the dissolvable matrices provide rapid dissolution and release of the one or more active agents in cold, cool, or room temperature liquid. This ability allows active agent delivery to and use of the matrices in numerous consumable beverages, e.g., juice, milk, and soda, which are normally served cold. Thereby, providing a palatable and desirable method for ingesting the one or more active ingredients.

The solid dissolvable matrices of the present disclosure have high mechanical stability and structural integrity (including being shelf-stable), rapidly dissolve in liquid, have predictable pore sizes and pore numbers, and low hygroscopicity. Many of these desirable properties are achieved by the choice of and amounts of excipients in a formulation; in some cases, the methods used to manufacture the dissolvable matrix helps provide the desirable properties. In some instances, dissolvable matrices are stable when stored for a period of time. In some instances, dissolvable matrices comprise a reproducible shelf life that meets a set of expected properties (e.g., industry or other standard). In some instances, dissolvable matrices retain function, dissolution rate, and handling properties over a period of time. In some instances, dissolvable matrices retain activity of one or more active agents over a period of time. In some instances, dissolvable matrices substantially retain structural integrity over a period of time. In some instances, dissolvable matrices do not substantially interact with primary packaging over a period of time. In some instances, dissolvable matrices do not substantially change moisture content over a period of time. In some instances, the period of time is at least 1 day, 5 days, 10 days, 30 days, 2 months, 6 months, 12 months, 18 months, 2 years, 3 years, or more than 3 years. In some instances, the period of time is no more than 1 day, 5 days, 10 days, 30 days, 2 months, 6 months, 12 months, 18 months, 2 years, 3 years, or no more than 5 years. In some instances, the period of time is 1-6, 1-12, 1-18, 1-24, 1-36, or 1-64 months. In some instances, dissolvable matrices are stored at a temperature of 0-25, 4-20, 4-15, 4-10, 0-10, −20 to 10, −20 to −4, or 1-8 degrees C.

### Matrix Excipients

A dissolvable matrix includes one or more excipients. Excipients are distinct from the active agents in that they do not provide a useful biological activity to a consumer; instead, they help provide desirable properties, as disclosed herein, to a dissolvable matrix. Five classes of excipients that are useful in the dissolvable matrices of the present disclosure include: pore-creating excipients, pore-size modifying excipients, mineral ion/mineral ion donors, hygroscopicity modifiers, and emulsifiers.

Some useful excipients regulate pore creation; examples of pore-creating excipients include but are not limited to, scaffolding agents (e.g., powdered cellulose), and blowing agents (e.g., a saponin, quillaja extract powder (e.g., from Quillaja saponaria), Yucca schidigera, agar, citric acid plus bicarbonate, azodicarbonamide, or other soaps/amphiphilic agents). In some instances, pore-creating excipients comprising an acid and a base (organic or mineral) are combined to produce a gas which contributes to pore creation. In some instances, a pore-creating excipient comprises a soap which traps air or other gases in the dissolvable matrix. In some instances, a dissolvable matrix comprises a plurality of pores. In some instances, a dissolvable matrix comprises one or more of a scaffolding agent, a blowing agent, and an active agent.

Generally, a dissolvable matrix can comprise at least 1%, 2%, 5%, 8%, 10%, 15%, 20%, or 30%, (w/w) of one or more pore-creating excipients relative to the entire dissolvable matrix, when in the condition provided to a user, e.g., in its shelf stable form and not the w/w in a formulation prior to the final product. In some instances, a dissolvable matrix comprises at least 1%, 2%, 5%, 8%, 10%, 15%, 20%, or 30%, (w/w) of one or more pore-creating excipients relative to the dissolvable matrix, not including the mass of the one or more active agents. The dissolvable matrix can comprise between about 1-30%, 2-25%, 5-25%, 10-25%, 10-20%, 15-20%, 15-25%, or 20-25% (w/w) of one or more pore-creating excipients; or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (w/w) of each pore-creating excipient in the one or more pore-creating excipients. In some instances, a dissolvable matrix comprises at least 1%, 2%, 5%, 8%, 10%, 15%, 20%, or 30%, (w/w) of one or more blowing agent excipients relative to the dissolvable matrix, not including the mass of the one or more active agents. The dissolvable matrix can comprise between about 1-30%, 2-25%, 5-25%, 10-25%, 10-20%, 15-20%, 15-25%, or 20-25% (w/w) of one or more blowing agent excipients; or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (w/w) of each blowing agent excipient in the one or more blowing agent excipients. In some instances, a dissolvable matrix comprises at least 1%, 2%, 5%, 8%, 10%, 15%, 20%, or 30%, (w/w) of one or more scaffolding excipients relative to the dissolvable matrix, not including the mass of the one or more active agents. The dissolvable matrix can comprise between about 1-30%, 2-25%, 5-25%, 10-25%, 10-20%, 15-20%, 15-25%, or 20-25% (w/w) of one or more scaffolding excipients; or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (w/w) of each scaffolding excipient in the one or more scaffolding excipients.

Quillaja extract powder can act like an emulsifier as well as pore-creating excipient, since it is saponin rich. Therefore, in a matrix comprising a water insoluble active agent(s), quillaja extract powder can be present in a higher amount, e.g., over 6% (w/w), whereas in a matrix comprising water soluble active agent(s), quillaja extract powder is present in about 1-2% (w/w) each relative to the entire dissolvable matrix, with respect to the final dissolvable matrix. In some instances, quillaja extract comprises saponins. In some instances, quillaja extract comprises at least 10%, 20%, 30%, 40%, 50%, 60%, 70%, 80%, or at least 90% saponins (w/w). In some instances, quillaja extract comprises 10-95%, 10-90%, 10-80%, 10-70%, 10-60%, 10-50%, 10-25%, 20%-90%, 20%-70%, 20%-50%, 30%-75%, 40%-85%, 40%-90%, 50%-75%, 50%-85%, 60%-90%, or 70%-95% saponins (w/w). In some instances, quillaja extract comprises at least 20% saponins (w/w).

The pore-creating excipient can comprise saponins. In some instances, a dissolvable matrix comprises 0.1-5%, 0.1-3%, 0.1-2%, 0.1-1%, 0.2-2%, 0.2-1%, 0.3-3%, 0.5-2%, 0.5-3%, 0.5-9%, 0.5-8%, 0.5-7%, 0.5-6%, 0.5-5%, 1-2.5%, 2%-9%, 2%-7%, 2%-10%, 5-10%, 3%-20%, 5-40%, or 10-40% saponins (w/w). In some instances, a dissolvable matrix comprises at least 0.05%, 0.1%, 0.2%, 0.3%, 0.5%, 1%, 1.5%, 2%, 2.5%, 3%, 4%, 5%, 6%, 8%, or at least 10% (w/w) saponins.

The pore-creating excipient can comprise powdered cellulose and quillaja extract powder. The amount of powdered cellulose can vary from about 8% to about 30% (w/w) and the amount of quillaja extract powder can vary from about 1% to about 7% (w/w); each with respect to the final dissolvable matrix. As examples, the amount of powdered cellulose can be about 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, or 30% (w/w) and the amount of quillaja extract powder can be about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, and 7% (w/w); each with respect to the final dissolvable matrix.

A formulation of dissolvable matrix 1, as disclosed herein, can comprise between about 16% and about 33% (w/w) of the pore-creating excipient relative to the entire dissolvable matrix. The pore-creating excipients may comprise about 16%, 17%, 18%, 19%, 20%, 21%, 22%, 23%, 24%, 25%, 26%, 27%, 28%, 29%, 30%, 31%, 32%, or 33% (w/w) with respect to the final dissolvable matrix. The pore-creating excipient can comprise powdered cellulose and quillaja extract powder. The amount of powdered cellulose can vary from about 15% to about 30% (w/w) and the amount of quillaja extract powder can vary from about 1% to about 3% (w/w); each with respect to the final dissolvable matrix. As examples, the amount of powdered cellulose can be about 15%, 16%, 17%, 18%, 19%, 20%, 21%, 22%, 23%, 24%, 25%, 26%, 27%, 28%, 29%, or 30% (w/w) and the amount of quillaja extract powder can be about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% (w/w). In specific examples, a formulation of dissolvable matrix 1 comprises about 17.73% powdered cellulose and about 1.48% quillaja extract powder (w/w), about 17.23% powdered cellulose and about 1.44% quillaja extract powder (w/w); about 18.83% powdered cellulose and about 1.57% quillaja extract powder (w/w), about 22.73% powdered cellulose and about 1.42% quillaj a extract powder (w/w), each relative to the final dissolvable matrix.

A formulation of dissolvable matrix 1a, as disclosed herein, can comprise between about 16% and about 33% (w/w) of the pore-creating excipient relative to the entire dissolvable matrix. The pore-creating excipients may comprise about 16%, 17%, 18%, 19%, 20%, 21%, 22%, 23%, 24%, 25%, 26%, 27%, 28%, 29%, 30%, 31%, 32%, or 33% (w/w) with respect to the final dissolvable matrix. The pore-creating excipient can comprise powdered cellulose and quillaja extract powder. The amount of powdered cellulose can vary from about 17% to about 20% (w/w) and the amount of quillaja extract powder can vary from about 1% to about 3% (w/w); each with respect to the final dissolvable matrix. As examples, the amount of powdered cellulose can be about 17%, 17.2%, 17.4%, 17.6%, 17.8%, 18%, 18.2%, 18.4%, 18.6%, 18.8%, 19%, 19.2%, 19.4%, 19.6%, 19.8%, or 20% (w/w) and the amount of quillaja extract powder can be about 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% (w/w). In specific examples, a formulation of dissolvable matrix 1a comprises about 17.73% powdered cellulose and about 1.48% quillaja extract powder (w/w), about 17.23% powdered cellulose and about 1.44% quillaja extract powder (w/w), or about 18.83% powdered cellulose and about 1.57% quillaja extract powder (w/w), each relative to the final dissolvable matrix.

A formulation of dissolvable matrix 2, as disclosed herein, can comprise between about 15% and about 30% (w/w) of the pore-creating excipient relative to the entire dissolvable matrix. The pore-creating excipients may comprise about 15%, 16%, 17%, 18%, 19%, 20%, 21%, 22%, 23%, 24%, 25%, 26%, 27%, 28%, 29%, or 30% (w/w) with respect to the final dissolvable matrix. The pore-creating excipient can comprise powdered cellulose and quillaja extract powder. The amount of powdered cellulose can vary from about 12% to about 20% (w/w) and the amount of quillaja extract powder can vary from about 3% to about 8% (w/w); each with respect to the final dissolvable matrix. As examples, the amount of powdered cellulose can be about 14%, 14.2%, 14.4%, 14.6%, 14.8%, 15%, 15.2%, 15.4%, 15.6%, 15.8%, 16%, 16.2%, 16.4%, 16.6%, 16.8%, 17%, 17.2%, 17.4%, 17.6%, 17.8%, or 18% (w/w) and the amount of quillaja extract powder can be about 5%, 5.2%, 5.4%, 5.6%, 5.8%, 6%, 6.2%, 6.4%, 6.6%, 6.8%, or 7% (w/w). In a specific example, a formulation of dissolvable matrix 2 comprises about 15.81% powdered cellulose and about 6.32% quillaja extract powder (w/w) relative to the final dissolvable matrix.

A formulation of dissolvable matrix 2a, as disclosed herein, can comprise between about 15% and about 30% (w/w) of the pore-creating excipient relative to the entire dissolvable matrix. The pore-creating excipients may comprise about 15%, 16%, 17%, 18%, 19%, 20%, 21%, 22%, 23%, 24%, 25%, 26%, 27%, 28%, 29%, or 30% (w/w) with respect to the final dissolvable matrix. The pore-creating excipient can comprise powdered cellulose and quillaja extract powder. The amount of powdered cellulose can vary from about 12% to about 20% (w/w) and the amount of quillaja extract powder can vary from about 2% to about 7% (w/w); each with respect to the final dissolvable matrix. As examples, the amount of powdered cellulose can be about 15%, 15.2%, 15.4%, 15.6%, 15.8%, 16%, 16.2%, 16.4%, 16.6%, 16.8%, 17%, 17.2%, 17.4%, 17.6%, 17.8%, or 18% (w/w) and the amount of quillaja extract powder can be about 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, or 6% (w/w). In a specific example, a formulation of dissolvable matrix 2a comprises about 16.71% powdered cellulose and about 4.18% quillaja extract powder (w/w) relative to the final dissolvable matrix.

Other useful excipients are classified as pore size modifiers (which are also capable of being emulsifier stabilizers); examples of pore size modifiers include but are not limited to fibers (e.g., oat fiber), cellulose or cellulose derivatives, (e.g., cellulose, microcrystalline cellulose), starches (e.g., tapioca starch), wheat bran, or lignins. Such pore size modifier/emulsifier stabilizers also act as bulking agents to a dissolvable matrix. In some instances, pore size modifiers have different density or volume properties relative to other components in the dissolvable matrix.

In some instances, the pore size modifier or emulsifier stabilizer has an average particle size of 10-300, 10-200, 10-100, 20-100, 40-80, 50-200, 100-200, 50-100, or 150-250 microns. In some instances, the pore size modifier or emulsifier stabilizer has a D50 (size in microns that splits the distribution of particles equally above and below a set diameter) of about 5, 10, 20, 25, 50, 75, 100, 125, 150, 200, or about 500 microns. In some instances, the pore size modifier or emulsifier stabilizer has a D50 (portion of particles with diameters smaller or larger than a size in microns) of 5-500, 10-200, 10-100, 5-25, 50-500, 75-125, 100-500, 200-500, 10-500, or 50-100 microns.

Generally, a dissolvable matrix can comprise at least 1%, 2%, 5%, 8%, 10%, 15%, or 20% (w/w) of one or more pore-size modifying excipients relative to the entire dissolvable matrix, when in the condition provided to a user, e.g., in its shelf stable form and not the w/w in a formulation prior to the final product. The dissolvable matrix can comprise between about 1-20%, 2-20%, 5-20%, 10-15%, 10-20%, or 15-20% (w/w) of one or more pore-size modifying excipients; or about 1%, 2%, 3%, 4%, 5%, 6%, 7%, 8%, 9%, 10%, or 11% (w/w) of each pore-size modifying excipient in the one or more pore-size modifying excipients.

A formulation of dissolvable matrix 1, as disclosed herein, can comprise between about 5% and 15% (w/w) of the pore-size modifying excipient relative to the entire dissolvable matrix. The pore-creating excipients may comprise about 5%, 5.2%, 5.4%, 5.6%, 5.8%, 6%, 6.2%, 6.4%, 6.6%, 6.8%, 7%, 7.2%, 7.4%, 7.6%, 7.8%, 8%, 8.2%, 8.4%, 8.6%, 8.8%, 9%, 9.2%, 9.4%, 9.6%, 9.8%, 10%, 10.2%, 10.4%, 10.6%, 10.8%, 11%, 11.2%, 11.4%, 11.6%, 11.8%, 12%, 12.2%, 12.4%, 12.6%, 12.8%, 13%, 13.2%, 13.4%, 13.6%, 13.8%, 14%, 14.2%, 14.4%, 14.6%, 14.8%, or 15% (w/w) with respect to the final dissolvable matrix. The pore-creating excipient can comprise microcrystalline cellulose. The amount of microcrystalline cellulose can vary from about 8% to about 13% (w/w). As examples, the amount of microcrystalline cellulose can be about 8%, 9%, 10%, 11%, 12%, or 13%. In specific examples, a formulation of dissolvable matrix 1 comprises about 11.37% microcrystalline cellulose (w/w), relative to the final dissolvable matrix. In some instances, a pore-creating excipient comprises a cellulose derivative (e.g., methylcellulose, hydroxypropyl cellulose, and carboxymethyl cellulose (CMC)).

A formulation of dissolvable matrix 1a, as disclosed herein, can comprise between about 5% and 10% (w/w) of the pore-size modifying excipient relative to the entire dissolvable matrix. The pore-creating excipients may comprise about 5%, 5.2%, 5.4%, 5.6%, 5.8%, 6%, 6.2%, 6.4%, 6.6%, 6.8%, 7%, 7.2%, 7.4%, 7.6%, 7.8%, 8%, 8.2%, 8.4%, 8.6%, 8.8%, 9%, 9.2%, 9.4%, 9.6%, 9.8%, 10% (w/w) with respect to the final dissolvable matrix. The pore-creating excipient can comprise microcrystalline cellulose. The amount of microcrystalline cellulose can vary from about 6% to about 8% (w/w). As examples, the amount of microcrystalline cellulose can be about 6%, 6.2%, 6.4%, 6.6%, 6.8%, 7%, 7.2%, 7.4%, 7.6%, 7.8%, 8% (w/w). In specific examples, a formulation of dissolvable matrix 1a comprises about 6.82%, 7.02%, or 7.45% microcrystalline cellulose (w/w), each relative to the final dissolvable matrix.

A formulation of dissolvable matrix 2, as disclosed herein, can comprise between about 1% and 13% (w/w) of the pore-size modifying excipient relative to the entire dissolvable matrix. The pore-size modifying excipients may comprise about 2%, 3%, 4%, 5%, 6%, 7%, 8%, 9%, 10%, 11%, 12%, and 13% (w/w) with respect to the final dissolvable matrix. The pore-size modifying excipient can comprise a fiber, e.g., oat fiber. The amount of oat fiber can vary from about 2.5% to about 4.5% (w/w) with respect to the final dissolvable matrix. As examples, the amount of oat fiber can be about 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, or 4.5% (w/w). In a specific example, a formulation of dissolvable matrix 2 comprises about 3.32% oat fiber relative to the final dissolvable matrix.

A formulation of dissolvable matrix 2a, as disclosed herein, can comprise between about 0.5% and 2.5% (w/w) of the pore-size modifying excipient relative to the entire dissolvable matrix. The pore-size modifying excipients may comprise about 0.5%, 1%, 1.5%, 2%, or 2.5% (w/w) with respect to the final dissolvable matrix. The pore-size modifying excipient can comprise tapioca starch. The amount of tapioca starch can vary from about 1% to about 2% (w/w) with respect to the final dissolvable matrix. As examples, the amount of tapioca starch can be about 1%, 1.1%, 1.2%, 1.3%, 1.4%, 1.5%, 1.6%, 1.7%, 1.8%, 1.9%, or 2% (w/w). In a specific example, a formulation of dissolvable matrix 2a comprises about 1.19% tapioca starch (w/w) relative to the final dissolvable matrix.

Alternately, a formulation of dissolvable matrix 2a, as disclosed herein, can comprise between about 1% and 13% (w/w) of the pore-size modifying excipient relative to the entire dissolvable matrix. The pore-size modifying excipients may comprise about 2%, 3%, 4%, 5%, 6%, 7%, 8%, 9%, 10%, 11%, 12%, and 13% (w/w) with respect to the final dissolvable matrix. The pore-size modifying excipient can comprise a fiber, (e.g., oat, brown rice, buckwheat, bulgur, millet, oatmeal, popcorn, quinoa, whole grain barley, whole-grain corn, whole oats/oatmeal, whole rye, whole wheat, rolled oats, or wild rice). The amount of fiber can vary from about 2.5% to about 4.5% (w/w) with respect to the final dissolvable matrix. As examples, the amount of fiber can be about 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, or 4.5% (w/w). The amount of oat fiber can vary from about 2.5% to about 4.5% (w/w) with respect to the final dissolvable matrix. In some instances, oat fiber is used. As examples, the amount of oat fiber can be about 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, or 4.5% (w/w). In a specific example, a formulation of dissolvable matrix 2a comprises about 3.32% oat fiber relative to the final dissolvable matrix.

Alternately, the pore-size modifying excipients may comprise about 8.5%, 9%, 9.5%, 10%, 10.5%, or 11% (w/w) with respect to the final dissolvable matrix. The pore-size modifying excipient can comprise tapioca starch. The amount of tapioca starch can vary from about 8% to about 10% (w/w) with respect to the final dissolvable matrix. As examples, the amount of tapioca starch can be about 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% (w/w). In a specific example, a formulation of dissolvable matrix 2a comprises about 9.46% tapioca starch (w/w) relative to the final dissolvable matrix.

Other useful excipients are classified as mineral ions or mineral ion donors. Such mineral ion/mineral ion donors in some instances reverse solubility (i.e., increased dissolution of the mineral at lower solvent temperatures) and allow for the disruption of the surface layer to increase the disintegration potential. In some instances, mineral ion/mineral ion donors work synergistically with the blowing agent to produces pores. For example, a mineral ion/mineral ion donors in some instances produces a gas upon mixing. Example of mineral ion donors include, but are not limited to, calcium, magnesium, potassium, sodium, iron, cobalt, zinc, copper, or manganese. Such mineral ions can be present as a phosphate, a carbonate, a sulfate, an iodide, a fluoride, or other counterion. In some instances a mineral ion comprises a carbonate and is mixed with an organic or inorganic acid. In some instances the mineral ion comprises a carbonate and/or citrate. In some instances, one or more active agents have specific pH requirements for use in a dissolvable matrix. In some instances, mineral ions are used to adjust the pH when formulating a dissolvable matrix described herein.

Generally, a dissolvable matrix can comprise between about 0.5% and about 20% (w/v), e.g., about or at least 0.5%, 1%, 2%, 3%, 4%, 5%, 6%, 7%, 8%, 9%, 10%, 11%, 12%, 13%, 14%, 15%, 16%, 17%, 18%, 19%, or 20% (w/w) of one or more mineral ions/mineral ion donors relative to the entire dissolvable matrix, when in the condition provided to a user, e.g., in its shelf stable form and not the w/w in a formulation prior to the final product. The dissolvable matrix can comprise between about 0.5-6%, 1-5%, 1.5-4%, 2-4%, 3-5%, or 4-5% (w/w) of one or more mineral ions/mineral ion donors; or about 0.25%, 0.5%, 0.8%, 1.1%, 1.4%, 1.7%, 2%, 2.3%, 2.6%, 2.9%, 3.2%, 3.5%, 3.8%, 4.1%, 4.4%, 4.7%, 5%, 5.3%, 5.6%, or 5.9% (w/w) of each mineral ions/mineral ion donors in the one or more mineral ions/mineral ion donors. The relative concentration of a mineral ion/mineral ion donors may be depend on the amount of lecithin that included a matrix. Without being bound by theory, mineral ions in some instances (e.g., calcium) may disrupt film forming properties of a matrix described herein. In some instances, mineral ions are added to matrices comprising a lecithinized botanical extract. In some instances, calcium ions are added to matrices comprising lecithinized botanical extract.

A formulation of dissolvable matrix 2a, as disclosed herein, can comprise between about 0.8% and 5% (w/w) of the mineral ions/mineral ion donors relative to the entire dissolvable matrix. The mineral ions/mineral ion donors may comprise about 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% (w/w) with respect to the final dissolvable matrix. The mineral ion/mineral ion donor may be calcium carbonate. The amount of calcium carbonate can vary from about 0.8% to about 3% (w/w) with respect to the final dissolvable matrix. As examples, the amount of calcium carbonate can be about 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3% (w/w). The amount of calcium carbonate can vary from about 3.5% to about 5% (w/w) with respect to the final dissolvable matrix. As examples, the amount of calcium carbonate can be about 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% (w/w). In specific examples, formulations of dissolvable matrix 2a comprise about 1.19% or about 4.19% calcium carbonate (w/w) each relative to the final dissolvable matrix.

An excipient may be a hygroscopicity modifier, designed to modulate the rate at which the matrix absorbs moisture in the air. In some instances, the hygroscopicity modifier comprises a hydrophilic substance. In some instances, the hygroscopicity modifier comprises an oil. In some instances, the hygroscopicity modifier comprises a vegetable oil. In some instances, the hygroscopicity modifier comprises an dairy-derived oil or fat. In some instances, the hygroscopicity modifier comprises a triglyceride. A hygroscopicity modifier can comprise a triglyceride oil such as a short-chain triglyceride (SCT) oil, a medium chain triglyceride (MCT) oil (e.g., MCT oil powder), or long chain triglyceride (LCT) oil. Examples of hygroscopicity modifiers herein include coconut oil, palm kernel oil, whole milk, and butter. In one example, a hygroscopicity modifier comprises a fatty acid. In one example, a hygroscopicity modifier is a C6-C12 fatty acid.

Generally, a dissolvable matrix can comprise at least 1%, 2%, 3%, 4%, or 5% (w/w) of one or more hygroscopicity modifiers relative to the entire dissolvable matrix, when in the condition provided to a user, e.g., in its shelf stable form and not the w/w in a formulation prior to the final product. The dissolvable matrix can comprise between about 1-5%, 2-5%, 2-4%, 3-5%, 3-4%, or 4-5% (w/w) of one or more mineral ions/mineral ion donors; or about 0.4%, 0.6%, 0.8%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, or 5% (w/w) of each mineral ions/mineral ion donors in the one or more mineral ions/mineral ion donors.

A formulation of dissolvable matrix 1, as disclosed herein, can comprise between about 1% and 5% (w/w) of the hygroscopicity modifiers relative to the entire dissolvable matrix. The hygroscopicity modifiers may comprise about 1%, 1.5%, 2%, 2.5%, 3%, 3.5%, 4%, 4.5%, or 5% (w/w) with respect to the final dissolvable matrix. The hygroscopicity modifier may be medium-chain triglyceride (MCT) oil powder. The amount of MCT oil powder can vary from about 0.8% to about 3% (w/w) with respect to the final dissolvable matrix. As examples, the amount of MCT oil powder can be about 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 0.2%, 4.3%, 4.4%, 4.5%, 4.6%, 4.7%, 4.8%, 4.9%, or 5% (w/w). In specific examples, formulations of dissolvable matrix 2a comprise about 4.26% MCT oil powder (w/w) each relative to the final dissolvable matrix.

A formulation of dissolvable matrix 1a, as disclosed herein, can comprise between about 1% and 5% (w/w) of the hygroscopicity modifiers relative to the entire dissolvable matrix. The hygroscopicity modifiers may comprise about 1%, 1.5%, 2%, 2.5%, 3%, 3.5%, 4%, 4.5%, or 5% (w/w) with respect to the final dissolvable matrix. The hygroscopicity modifier may be MCT oil powder. The amount of MCT oil powder can vary from about 0.8% to about 3% (w/w) with respect to the final dissolvable matrix. As examples, the amount of MCT oil powder can be about 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4% (w/w). In specific examples, formulations of dissolvable matrix 2a comprise about 2.96% or about 3.14% MCT oil powder (w/w) each relative to the final dissolvable matrix.

Other examples of excipients are emulsifiers. In some instances, emulsifier include but are not limited to carboxymethyl cellulose gum (CMC gum), mustard, soy and egg lecithin, mono- and diglycerides, polysorbates, carrageenan, guar gum, xanthan gum, or canola oil. An emulsifier can affect the inherent homogeneity of the ink, and the overall dispersion and suspension of the material within an aqueous form.

A formulation of dissolvable matrix 2, as disclosed herein, can comprise between about 0.05% and 1.0% (w/w) of an emulsifier relative to the entire dissolvable matrix. The emulsifier may comprise about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% (w/w) carboxymethyl cellulose gum (CMC) gum with respect to the final dissolvable matrix. The emulsifier may be CMC gum. The amount of CMC gum can vary from about 0.05% to about 0.25% (w/w) with respect to the final dissolvable matrix. As examples, the amount of MCT oil powder can be about 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, or 0.25% (w/w). In a specific example, a formulation of dissolvable matrix 2 comprises about 0.16% CMC gum (w/w) relative to the final dissolvable matrix.

A formulation of dissolvable matrix 2a, as disclosed herein, can comprise between about 0.05% and 1.0% (w/w) of an emulsifier relative to the entire dissolvable matrix. The emulsifier may comprise about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% (w/w) carboxymethyl cellulose gum (CMC) gum with respect to the final dissolvable matrix. The emulsifier may be CMC gum. The amount of CMC gum can vary from about 0.05% to about 0.25% (w/w) with respect to the final dissolvable matrix. As examples, the amount of MCT oil powder can be about 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, or 0.25% (w/w). In a specific example, a formulation of dissolvable matrix 2 comprises about 0.16% CMC gum (w/w) relative to the final dissolvable matrix. However, in formulations of matrix 2a, which includes lecithin, lower amounts of emulsifiers are needed in a dissolvable matrix. This is because the lecithin comprises phosphatidylcholine, which is an emulsifier.

Any of the dissolvable matrices disclosed herein may comprise an excipient that comprises a humectant. Humectants can be present in 2-20% 2-15%, 2-10%, 2-5%, 3-5%, 3-10%, 3-6%, 4-8%, 4-15%, 5-10%, 5-15%, 5-20%, 6-10%, 6-15%, 8-15%, 8-20%, 10-20%, 12-20%, 15-20%, 15-30% (w/w) with respect to the final dissolvable matrix. In some instances humectants are present in about 2%, 3%, 4%, 5%, 6%, 7%, 8%, 9%, 10%, 11%, 12%, 13%, 14%, or 15% (w/w) with respect to the final dissolvable matrix. In specific examples, a formulation of dissolvable matrix 1a comprises about 3.14% humectant (w/w), a formulation of dissolvable matrix 1a comprises about 4.43% humectant (w/w), a formulation of dissolvable matrix 1a comprises about 5.74% humectant (w/w), a formulation of dissolvable matrix 2 comprises about 12.65% humectant (w/w), and a formulation of dissolvable matrix 2a comprises about 7.16% humectant (w/w), each relative to the final dissolvable matrix. In some instances glycerin is used as an excipient. Glycerin can be present in 2-20% 2-15%, 2-10%, 2-5%, 3-5%, 3-10%, 3-6%, 4-8%, 4-15%, 5-10%, 5-15%, 5-20%, 6-10%, 6-15%, 8-15%, 8-20%, 10-20%, 12-20%, 15-20%, 15-30% (w/w) with respect to the final dissolvable matrix. In some instances glycerin is present in about 2%, 3%, 4%, 5%, 6%, 7%, 8%, 9%, 10%, 11%, 12%, 13%, 14%, or 15% (w/w) with respect to the final dissolvable matrix. In specific examples, a formulation of dissolvable matrix 1a comprises about 3.14% glycerin (w/w), a formulation of dissolvable matrix 1a comprises about 4.43% glycerin (w/w), a formulation of dissolvable matrix 1a comprises about 5.74% glycerin (w/w), a formulation of dissolvable matrix 2 comprises about 12.65% glycerin (w/w), and a formulation of dissolvable matrix 2a comprises about 7.16% glycerin (w/w), each relative to the final dissolvable matrix. In some instances humectants include but are not limited to glycerin, alpha hydroxyl acids (e.g., glycolic acid, lactic acid), sodium pyrrolidine carboxylic acid, propylene glycol, butylene glycol, hyaluronic acid, urea, panthenol, aluminum lactate, sodium lactate, gelatin, sorbitol, or honey.

Any of the dissolvable matrices disclosed herein may comprise a film forming excipient. In some instances, film forming excipients generate a film on the outside of a dissolvable matrix. In some instances a film forming excipient is present in 1-5%, 1-4%, 1-3%, 1-2%, 2-8%, 2-5%, 2-4%, or 2-5%, 3-5%, or 4-5% (w/w) with respect to the final dissolvable matrix. In some instances a film forming excipient is present in about 1%, 2%, 3%, 4%, or 5% (w/w), e.g., about 2%, 2.2%, 2.4%, 2.6%, 2.8%, 3%, 3.2%, 3.4%, 3.6%, 3.8%, 4%, 4.2%, 4.4%, 4.6%, 4.8%, or 5% (w/w) with respect to the final dissolvable matrix. In specific examples, a formulation of dissolvable matrix 1a comprises about 3.81% film forming excipient (w/w), a formulation of dissolvable matrix 1a comprises about 3.59% film forming excipient (w/w), a formulation of dissolvable matrix 1a comprises about 4.16% film forming excipient (w/w), a formulation of dissolvable matrix 2 comprises about 3.64% film forming excipient (w/w), and a formulation of dissolvable matrix 2a comprises about 2.39% film forming excipient (w/w), each relative to the final dissolvable matrix. In some instances, film-forming excipients include but are not limited to starch, polymerized rosin, pullulan, sodium alginate, Pectin, gelatin, and maltodextrins, Polyvinyl alcohol, hydroxy propyl methyl cellulose, sodium carboxy methyl cellulose, polyvinyl pyrrolidone, proteins (e.g., collagen) and hydroxy propyl cellulose. In some instances, pullulan is used as an excipient. For example, pullulan acts as a film former to a dissolvable matrix. In some instances pullulan is present in 1-5%, 1-4%, 1-3%, 1-2%, 2-8%, 2-5%, 2-4%, or 2-5%, 3-5%, or 4-5% (w/w) with respect to the final dissolvable matrix. Pullulan is present in about 1%, 2%, 3%, 4%, or 5% (w/w), e.g., about 2%, 2.2%, 2.4%, 2.6%, 2.8%, 3%, 3.2%, 3.4%, 3.6%, 3.8%, 4%, 4.2%, 4.4%, 4.6%, 4.8%, or 5% (w/w) with respect to the final dissolvable matrix. In specific examples, a formulation of dissolvable matrix 1a comprises about 3.81% pullulan (w/w), a formulation of dissolvable matrix 1a comprises about 3.59% pullulan (w/w), a formulation of dissolvable matrix 1a comprises about 4.16% pullulan (w/w), a formulation of dissolvable matrix 2 comprises about 3.64% pullulan (w/w), and a formulation of dissolvable matrix 2a comprises about 2.39% pullulan (w/w), each relative to the final dissolvable matrix.

Additional classes of excipients useful in dissolvable matrices of the present invention include bulking agents and/or flowing agents.

A dissolvable matrix can include other ingredients and/or excipients, including one or more of polymers, defoamers, flow aides, flavor enhancers, rheological modifiers, humectants, waxes, and the like and other components that are utilized to print a layer from an ink, such as dyes, pigments, etc. Exemplary polymers in some instances are water soluble, water swellable, or water insoluble. They include but are not limited by, ethyl cellulose, polyacrylic acid, methyl cellulose, polyethylene oxide, guar gum, xanthan gum, gum Arabic, polyvinyl alcohol, sodium alginate, water-soluble hydroxypropylmethyl cellulose, hydroxyethyl cellulose, hydroxypropyl cellulose, polyvinyl pyrrolidone, carboxymethyl cellulose, sodium carboxy methyl cellulose, methyl cellulose, polyvinyl alcohol, xanthan gum, various starches such as corn starch, rice starch etc. Defoamers may include, but are not limited thereto, alcohol or polysiloxane type defoamers both in water and alcohol. Flow aids may contain food grade glycols and polyglycols, xylitol, glycerol. Waxes may include, but are not limited to, paraffin or carnauba waxes. Humectants may include, but are not limited to, all molecular weight polyethylene glycols and propylene glycols, xylitol, glycerol sugars and starches. Rheology modifiers may include, but are not limited to, sodium salts of an acrylic polymer, various starches and gums. Colorants may also be used to tint printed compositions to specific colors.

### Properties of Dissolvable Matrices

Desirable properties of the solid dissolvable matrices of the present disclosure include high mechanical stability and structural integrity (including being shelf-stable), rapidly dissolution in liquid, predictable pore sizes and pore numbers, and low hygroscopicity. Many of these desirable properties are achieved by the choice of and amounts of excipients in a formulation, as disclosed above. In some cases, the methods used to manufacture the dissolvable matrix helps provide the desirable properties.

### Mechanical Stability

Dissolvable matrices described herein may be sufficiently robust in terms of shelf stability and/or mechanical stability. In some instances, matrices have sufficient stiffness to handle and/or orally ingest and/or place in a food product, such as a beverage, without sagging to a degree that makes handling difficult. Matrices are formulated to not be brittle, such that they resist crumbling when handled under normal conditions.

### Dissolution

Dissolvable matrices described herein have specific dissolution profiles in a solvent. Solvents can be substantially or mostly pure (e.g., water), but more complex solvents are also contemplated. Solvents include but are not limited to juice, water, tea, milk, coffee, carbonated beverage, fermented beverages (beer, wine, kombucha), soda, or other solvent. A matrix may dissolve (or disintegrate) in a solvent with no or minimal mechanical stirring. A matrix may dissolve in a solvent by active mechanical stirring, such as that achievable by a hand stirring with a spoon, or even a blender or other electrical mixing device. Dissolutions rates of the matrix may be affected by solvent volumes, solvent temperatures, solvent pH, time, or other property of the solvent or the matrix. The surface area of the matrix may affect dissolution rates. In some instances, a larger surface area results in faster dissolution. In some instances, surface is controlled by the shape of the matrix, or pores therein.

Dissolvable matrices are configured to dissolve (or disintegrate) in a certain temperature of water (or other aqueous solution) after a certain period of time. In some instances, immersion comprises contacting dissolvable matrices with excess water (or other aqueous solution). In some instances excess water comprises an amount of water (or other aqueous solution) at least 2, 3, 5, 10, 20, 50, or at least 100 times the mass of the dissolvable matrix. In a non-limiting example, a dissolvable matrix (e.g., 1.5 inch×1.5 inch×200 micron thick matrix) dissolves within one minute when immersed in 200 mL water at 4 degrees C. with mechanical stirring. In some instances, dissolution conditions are measured using mechanical stirring. In some instances, dissolution conditions are measured without the use of mechanical stirring. In some instances, dissolvable matrices disintegrate without the use of mechanical stirring. In some instances, dissolution is controlled by formulation (e.g., the choice and amount of excipients or additional components, and whether the active agent(s) place the matrix in a category 1, 1a, 2, or 2a class), the size/surface area of the matrix, the size/number of pores, and so forth. The dissolvable matrix may be configured to dissolve in water (or other aqueous solution) having a temperature of no more than 30° C. in less than 1, 2, 5, 10, 15, 20, 30, 45, 60, 90, or 120 seconds. The dissolvable matrix may be configured to dissolve in water (or other aqueous solution) having a temperature of no more than 30° C. in less than 10 seconds. The dissolvable matrix may be configured to dissolve in water (or other aqueous solution) having a temperature of no more than 20° C. in less than 1, 2, 5, 10, 15, 20, 30, 45, 60, 90, or 120 seconds. The dissolvable matrix may be configured to dissolve in water (or other aqueous solution) having a temperature of no more than 20° C. in less than 60 seconds. The dissolvable matrix may be configured to dissolve in water (or other aqueous solution) having a temperature of no more than 10° C. in less than 1, 2, 5, 10, 15, 20, 30, 45, 60, 90, or 120 seconds. The dissolvable matrix may be configured to dissolve in water (or other aqueous solution) having a temperature of no more than 10° C. in less than 60 seconds. The dissolvable matrix may be configured to dissolve in water (or other aqueous solution) having a temperature of no more than 5° C. in less than 1, 2, 5, 10, 15, 20, 30, 45, 60, 90, or 120 seconds. The dissolvable matrix may be configured to dissolve in water (or other aqueous solution) having a temperature of no more than 5° C. in less than 60 seconds. The dissolvable matrix may be configured to dissolve in water (or other aqueous solution) having a temperature of about 0° C. in less than 1, 2, 5, 10, 15, 20, 30, 45, 60, 90, or 120 seconds. The dissolvable matrix may be configured to dissolve in water (or other aqueous solution) having a temperature of about 0° C. in less than 60 seconds. The dissolvable matrix may be configured to dissolve in no more than 8 oz of water (or other aqueous solution) having a temperature of no more than 20° C. in less than 60 seconds. The dissolvable matrix may be configured to dissolve in no more than 8 oz of water having a temperature of no more than 20° C. in less than 30 seconds. The dissolvable matrix may be configured to dissolve in an aqueous solution having a pH of about 2 to 10.

Dissolvable matrices described herein may have different surface areas which may influence dissolution. In some instances, the dissolvable matrix surface area is not measured to include pores (i.e., treated as a flat external surface based on overall shape). In some instances, a dissolvable matrix has a surface area of at least 100, 200, 500, 800, 1000, 2000, 5000, 8000, 10,000, 12,000, 15,000, or at least 20,000 mm². A dissolvable matrix may have the size and dimensions of an 8½″ by 11″ sheet. A dissolvable matrix may have a surface area of about 100, 200, 500, 800, 1000, 2000, 5000, 8000, 10,000, 12,000, 15,000, or about 20,000 mm². In some instances, a dissolvable matrix has a surface area of 100-10,000, 100-20,000, 200-10,000, 500-10,000, 1000-10,000, 5000-10,000, 9000-15,000, or 7000-20,000 mm².

Additional components to regulate dissolution may be added to a formulation. Examples of such additional component are starches. A formulation may additionally or alternatively comprise one or more of polyvinyl alcohol, polysaccharides (e.g., Pullulan), sodium alginate, carrageenan, xanthan gum, or guar gum to regulate dissolution.

A dissolvable matrix may be configured such that the dissolution of the active or other ingredients (e.g., sweeteners and flavors) within the dissolvable matrix may be released over a period of time. The period of dissolution or dispersion may be adjusted based on the amount of starch, as an example, such as a slower period of dissolution or dispersion when more starch is used and quicker dissolution or dispersion when less starch is used (or vice versa). In some instances, a binder may be added to the dissolvable matrix to maintain the structural integrity of the substances therein. Binders may include one or more of polysaccharides (e.g., Pullulan,) sodium alginate, etc. In some instances, the entire dissolvable matrix and/or individual layers of the dissolvable matrix may include be subjected to micro-scoring and/or pinholes. By doing such, the surface area of the dissolvable matrix and/or layers is increased, thereby allowing for faster dissolution/dispersion.

Similar to the controlled release of the layers, described above, the support substrate may be configured such that the dissolution or dispersion of the support substrate may be performed over a period of time. In this regard, additional components such as starches may be mixed with polyvinyl alcohol and into one of the materials for generating the printable support substrate such as carrageenan, xanthan gum, guar gum, etc. The period of dissolution or dispersion may be adjusted by adjusting the formulation of the composition. For instance, based on the ingredients contained in the composition, such as the amount starch, the dissolution or dispersion rate may be adjusted. In one such example, a slower period of dissolution or dispersion may occur when more starch is used and quicker dissolution or dispersion when less starch is used. In some instances, a binder may be added to the dissolvable matrix to maintain the structural integrity of the substances therein. In some instances, the support substrate may include supplements or other active ingredients.

### Pore Sizes

Dissolvable matrices described herein comprise a plurality of pores, which form during the manufacturing process, e.g., as a result a blowing agents, which promote pore formation and help create structure in the matrix. Prior to curing, the blowing agents incorporate or trap air (or other gas, e.g., CO₂), e.g., create a froth, from a liquid composition comprising the active agents and excipients. After curing (i.e., when the matrix is dried and in its final product state), such pores create a void in the matrix (e.g., a volume that is not occupied by solid matter). Also, these pores contribute to improved handling and dissolution properties of the matrix. The voids increase the surface area contactable by a liquid solvent when dissolving a matrix. In some instances, the matrix comprises at least 0.5%, 1%, 1.5%, 2%, 3%, 5%, 10%, 15%, 20%, 25%, 30%, 35%, 40%, 45%, 50%, 55%, 60%, 65%, or at least 70% void space (v/v). In some instances, the matrix comprises about 0.5%, 1%, 1.5%, 2%, 3%, 5%, 10%, 15%, 20%, 25%, 30%, 35%, 40%, 45%, 50%, 55%, 60%, 65%, or about 70% void space (v/v). In some instances, the matrix comprises no more than 0.5%, 1%, 1.5%, 2%, 3%, 5%, 10%, 15%, 20%, 25%, 30%, 35%, 40%, 45%, 50%, 55%, 60%, 65%, or no more than 70% void volume/space (v/v). In some instances, the matrix comprises a void volume of 1-70%, 1-60%, 1-50%, 1-40%, 1-30%, 1-25%, 2-70%, 2-50%, 2-30%, 3-75%, 3-50%, 3-30%, 3-25%, 4-40%, 4-30%, 4-25%, 4-20%, 4-10%, 5-50%, 5-75%, 5-25%, 10-70%, 10-50%, 25-75%, or 50-75%. In some instances, the pore size is expressed as the longest linear dimension of a pore (e.g., longest cross-sectional length). In some instances, the average pore size is about 0.1, 0.2, 0.3, 0.5, 1, 2, 3, 5, 10, 20, 50, or about 100 microns. In some instances, the average pore size is no more than 0.1, 0.2, 0.3, 0.5, 1, 2, 3, 5, 10, 20, 50, or no more than 100 microns. In some instances, the average pore size is 0.1-50, 0.2-20, 0.5-50, 1-20, 1-50, 1-100, 5-100, 10-100, 10-200, 50-200, 100-200, 100-300, 150-300, 10-50, or 5-50 microns. In some instances, the median pore size is no more than 0.1, 0.2, 0.3, 0.5, 1, 2, 3, 5, 10, 20, 50, or no more than 100 microns. In some instances, the median pore size is 0.1-50, 0.2-20, 0.5-50, 1-20, 1-50, 1-100, 5-100, 10-100, 10-200, 50-200, 100-200, 100-300, 150-300, 10-50, or 5-50 microns. In some instances, pores are approximated as spherical. In some instances, pore size is measured as the longest cross-sectional diameter of the pore. In some instances, the pore size is expressed as a cross-sectional area of a pore. In some instances, the average cross-sectional area of pores is 1000-25,000, 1000-20,000, 1000-10,000, 1000-5000, 1500-12,000, 1500-7000, 1500-5000, 2500-25,000, 5000-25,000, 10,000-25,000, 15,000-25,000, or 7000-12000 square microns. In some instances, the average cross-sectional area of pores has a standard deviation of 1000-25,000, 1000-20,000, 1000-10,000, 1000-5000, 1500-12,000, 1500-7000, 1500-5000, 2500-25,000, 5000-25,000, 10,000-25,000, 15,000-25,000, or 7000-12000 square microns.

Dissolvable matrices may have a pore density. In some instances, the pore density is expressed in two dimensions as pores per unit area. In some instances, the pore density is expressed in three dimensions as pores per unit volume. In some instances, the pore density is between about 1-500, 1-250, 1-200, 1-150, 1-100, 10-500, 10-300, 10-200, 10-100, 25-150, 25-250, 50-100, 50-250, or between about 50-500 pores per square mm. In some instances, the pore density is at least 1, 2, 5, 10, 15, 20, 25, 50, 60, 70, 80, 90, 100, 125, 150, 175, 200, 225, 250, 300, 350, or at least 400 pores per square mm. In some instances, a dissolvable matrix comprises at least 1, 5, 10, 50, 100, 500, 1000, 5000, 10,000, or at least 50,000 pores per cubic mm. In some instances, a dissolvable matrix comprises about 1, 5, 10, 50, 100, 500, 1000, 5000, 10,000, or about 50,000 pores per cubic mm. In some instances, a dissolvable matrix comprises no more than 1, 5, 10, 50, 100, 500, 1000, 5000, 10,000, or no more than 50,000 pores per cubic mm. In some instances, a dissolvable matrix comprises 1-100,000, 1-50,000, 1-10,000, 1-5000, 1-1000, 1-100, 10-100, 10-1000, 50-500, 50-1000, 100-10,000, 100-5,000, 1000-100,000, 1000-50,000, 100-10,000, 100-1000, or 500-5000 pores per cubic mm.

The porosity of a dissolvable matrix described herein may be measured analytically. In some instances, a dissolvable matrix is analyzed using microscopy. In some instances, microscopy comprises optical, electron, scanning probe, or x-ray spectroscopy. In some instances, a matrix is mounted directly to a scanning electron microscopy (SEM) stud. In some instances, microscopy comprises SEM. In some instances, SEM is performed at 3.0 kV. In some instances, a dissolvable matrix is frozen (e.g., with liquid nitrogen or other agent) and fractured to produce a cross-section for mounting and analysis. In some instances, images are obtained from different planes of a dissolvable matrix. In some instances, images are obtained from one or more of x-y and z planes, where the x-y plane is a plane that corresponds with or is parallel to a principal surface of a matrix or disk, while the z plane is perpendicular to the x-y plane. In some instances, a dissolvable matrix is cut or fractured at room temperature to produce a cross-section for analysis. Images may then be obtained from the cross-section, and analyzed using a computer algorithm. The analysis in some instances measures void volume (% of total focal area, assuming spherical pores), average pore size, pore distribution, surface area per volume (taking into account the pores, and/or other measurement). In some instances, the x-y plane corresponding to the top (distal to a curing surface) of the dissolvable matrix is imaged. In some instances, one or more computer transformation steps are utilized to generate data for pores. In some instances, steps comprise one or more of opening an SEM image file, setting or defining the scale of the image, duplicating the image, converting the image to 8-bit grayscale type, setting a color threshold to represent pores in original image, processing or touching up the image based on features in the original image, sorting particles by minimum pore size, and logging data. Calculation of values in some instances comprises removal of one or more SEM images which do not meet minimum quality standards. In some instances, at least 1, 2, 3, 4, 5, 6, 7, 8, 9, 10, or more than 10 images are analyzed. In some instances, 1-10, 1-6, 2-6, 2-4, or 6-12 images are analyzed, e.g., using ImageJ or a similar program.

### Hygroscopicity

Dissolvable matrices described herein may have sufficiently low hygroscopicity to allow facile handling under various humidity conditions. In some instances, standard temperature and pressure (STP) used for measuring hygroscopicity were 0 degrees C. at 1 atm. In some instances, temperature and pressure used for measuring hygroscopicity were 20 degrees C. at 1 atm. In some instances, absorbance of moisture is measured as an initial rate. In some instances, the initial rate is measured during about the first minute, two minutes, five minutes, 10 minutes, 15 minutes, 30 minutes, hour, two hours, three hours, four hours, or 12 hours. In some instances, the initial rate is measured during 0-30 minutes, 0-60 minutes, 0-2 hours, 0-3 hours, 0-4 hours, 0-5 hours, or 0-6 hours. In some instances, the initial rate is no more than 0.1, 0.05, 0.04, 0.03, 0.02, 0.015, 0.010, or no more than 0.005% per minute. In some instances, the initial rate (measured for the first 4 hours) is no more than 0.1, 0.05, 0.04, 0.03, 0.02, 0.015, 0.010, or no more than 0.005% per minute. In some instances, the initial rate is 0.010-0.1, 0.010-0.2, 0.010-0.3, 0.010-0.04, 0.010-0.05, 0.010-0.07, 0.010-0.080, 0.02-0.05, 0.01-0.03, 0.015-0.03, or 0.03-0.05% per minute. In some instances, the initial rate is 0.010-0.1, 0.010-0.2, 0.010-0.3, 0.010-0.04, 0.010-0.05, 0.010-0.07, 0.010-0.080, 0.02-0.05, 0.01-0.03, 0.015-0.03, or 0.03-0.05% per minute for the first 4 hours. A matrix may absorb less than 1% (w/w) moisture per min at 50% humidity, e.g., less than 0.1%, 0.01%, or 0.01% (w/w) moisture per min at 50% humidity at standard temperature and pressure. A matrix may absorb less than 1% (w/w) moisture per min at 75% humidity, e.g., less than 0.1%, 0.01%, or 0.01% (w/w) moisture per min at 75% humidity at standard temperature and pressure. A matrix may absorb less than 1% (w/w) moisture per min at 90% humidity, e.g., less than 0.1%, 0.01%, or 0.01% (w/w) moisture per min at 90% humidity at standard temperature and pressure. A matrix may absorb less than 1% (w/w) moisture per min at 50% humidity, e.g., less than 0.1%, 0.01%, or 0.01% (w/w) moisture per min at 50% humidity at standard temperature and pressure. A matrix may absorb less than 1% (w/w) moisture per min at 75% humidity, e.g., less than 0.1%, 0.01%, or 0.01% (w/w) moisture per min at 75% humidity at standard temperature and pressure. A matrix may absorb less than 1% (w/w) moisture per min at 90% humidity, e.g., less than 0.1%, 0.01%, or 0.01% (w/w) moisture per min at 90% humidity at standard temperature and pressure.

A dissolvable matrix may be generated such that the matrix resists reaching a threshold water content (e.g., water activity or percent moisture). In some instances this facilitates handling of the matrix and/or removal of the matrix from a substrate or support (e.g., foil, wrapper, or other substrate). In some instances, a dissolvable matrix comprises a water activity of no more than 0.5, 0.45, 0.4, 0.35, 0.3, 0.25 0.2, or no more than 0.1. In some instances, a dissolvable matrix comprises a water activity of no more than 0.5, 0.45, 0.4, 0.35, 0.3, 0.25 0.2, or no more than 0.1 under conditions comprising 20 degrees C., 1 atm, and 45% relative humidity. In some instances, a dissolvable matrix comprises a water activity of no more than 0.5, 0.45, 0.4, 0.35, 0.3, 0.25 0.2, or no more than 0.1 under conditions comprising 20 degrees C., 1 atm, and 45% relative humidity after no more than 2 hours. In some instances, a dissolvable matrix comprises a water activity of no more than 0.5, 0.45, 0.4, 0.35, 0.3, 0.25 0.2, or no more than 0.1 under conditions comprising 20 degrees C., 1 atm, and 45% relative humidity after no more than 4 hours. In some instances, a dissolvable matrix comprises a water activity of no more than 0.5, 0.45, 0.4, 0.35, 0.3, 0.25 0.2, or no more than 0.1 under conditions comprising 20 degrees C., 1 atm, and 45% relative humidity after no more than 1 hour.

### Active Agents

A matrix herein can include one or more active agents, e.g., 1, 2, 3, 4, 5, 6, 7, 8, 9 or 10 active agents.

Such active agents include but are not limited to pharmaceuticals, nutraceuticals, plant, animal, or fungal extracts, or other active agents that promote health. Active agents can comprise prebiotics, fruit or vegetable extracts, minerals, amino acids, vitamins, lecithin, or another active agent. Active agents, such as prebiotics for example, can promote gut flora or microbiota health. Prebiotics include, but are not limited to, bacteriophages, polyphenols, and other. Other examples of active agents include sleep enhancers configured to promote sleep (quantity, quality, etc.), stimulants such as caffeine, steroids and the like. Active agents can be immunity enhancers, configured to promote enhanced immunity from diseases or conditions. In some instances, the active agent comprises a pharmaceutical or nutraceutical.

The percentage of active agents in some instances is measured as a percent dry weight after curing. The percentage of active agents in some instances is measured as a percent weight prior to mixing.

Dissolvable matrices comprise a surprisingly high percentage of active agents(s). A dissolvable matrix of the present disclosure may comprise between about 35% and 65% (w/w) of active agent(s) relative to the entire dissolvable matrix. The total active agents may comprise about 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% (w/w) with respect to the final dissolvable matrix. A dissolvable matrix may comprise one to ten active agents. The amount of each active agent in a matrix can be the same or differ. One active agent may be present in a greater amount the other active agents, e.g., 2-fold, 5-fold, 10-fold, 50-fold 100-fold, and 200-fold more; one active agent may be present in a lesser amount that the other active agents. One active agents may comprise less than 1% of the total weight of a dissolvable matrix, e.g., about 0.1%, 0.2%, 0.5%, or 0.8%; another active agent may comprise more than 30% of the total weight of a dissolvable matrix, e.g., about 30%, 31%, 32%, 33%, 34%, and 35%. In some cases, an active agent in a dissolvable matrix can be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% (w/w) relative to relative to the entire dissolvable matrix.

Active agents can be present in at least 10, 20, 30, 40 50, 100, 200, 500, 800, 1000, 2000, or at least 5000 mg per 10 cm² of a matrix. In some instances, active agent(s) are present in 500-800, 100-800, 50-200, 100-500, 200-800, 500-1000, or 500-1500 mg per 10 cm² of a matrix.

When an active agent is a prebiotic, the prebiotic can comprise a bacteriophage component. The bacteriophage component can comprise one or more lytic bacteriophages, such as, e.g., Siphoviridae or Myoviridae family, or more specifically, LH01-Myoviridae, LL5-Siphoviridae, T4D-Myoviridae, or LL12-Myoviridae. Alternatively, or in addition to, a bacteriophage component is one that accelerates growth of one or more of B. bifidum; B breve; B. animalis subsp. lactis; B. longum; L. acidophilus; L. paracasei; L. plantarum; L. rhamnosus; or B. subtilis. A bacteriophage component can be one that supports increases in the concentration of butyrate-producing Eubacteria, decreases the concentration of Clostridium perfringens, or decreases interleukin 4 (IL-4) cytokine. A bacteriophage can be one targets one or more pathogenic bacteria. In some instances, the bacteriophage targets Escherichia coli and related species. A matrix described herein can comprise between about 1-5%, 0.5-5%, 0.5-10%, 0.1-5%, 1-10%, 1.5-8%, 2-5%, or 2-6% (w/w) of a bacteriophage component or alternatively about 0.5%, 0.8%, 1%, 1.5%, 2%, 2.5%, 3%, 3.5%, 4% or about 5% of a prebiotic component (w/w) relative to the entire dissolvable matrix. In one example, a dissolvable matrix comprises about 2.9% the bacteriophage component (w/w) relative to the entire dissolvable matrix. PreforPro is an illustrative prebiotic comprising a bacteriophage component.

A dissolvable matrix can comprise one or more extracts. Examples of extracts contemplated herein include a fruit extract, a vegetable extract, or a tea leaf extract. Any of the extracts herein can comprise one or more polyphenols.

Examples of fruit extracts include blueberry, cherry, pomegranate, strawberry, banana, coconut, elderberry, currant, grape skin, pineapple, mango, papaya, kiwi, orange, paw, mangostene, acai, lemon, lime, grapefruit, cumquat, bergamot, tomato, and apple extract. A blueberry extract herein can be derived from, e.g., Vaccinium spp. such as Vaccinium alaskaense How; Vaccinium ovaliforium Sm; Vaccinium membranaceum L.; Vaccinium uliginosum L.; or Vaccinium cespitosum Mich X A matrix described herein can comprise about 1-15%, 1-20%, 5-25%, 10-20%, 10-25%, 15-25%, 5-10%, or 8-18% (w/w) of a blueberry extract or about 5%, 7%, 9%, 10%, 12%, 15%, 16%, 17%, 20%, 22%, 25%, or about 30% of a blueberry extract (w/w) each relative to the entire dissolvable matrix. A pomegranate extract (e.g., a pomegranate polyphenol powder) herein can derived from Punica spp. such as Punica granatum. Examples of pomegranate be, e.g., ellagitannins or punicalagins. A matrix described herein can comprise about 0.5-15%, 1-10%, 5-10%, 5-15%, 2-17%, 4-16%, 2-8%, or 2-15% (w/w) of a pomegranate extract or about 4%, 5%, 6%, 7%, 8%, 9%, 10%, 12%, 14%, 15%, or about 20% of a pomegranate extract (w/w) each relative to the entire dissolvable matrix. In one example, a dissolvable matrix comprises about 15.69% blueberry powder and about 7.85% pomegranate extract (w/w) each relative to the entire dissolvable matrix. An illustrative cherry extract is acerola (cherry) powder. In one example, a dissolvable matrix comprises about 1.63% acerola powder (w/w) relative to the entire dissolvable matrix.

A vegetable extract can be any one or more of the following: turmeric, beet root, broccoli, daikon, garlic, chicory, asparagus, cucumber, celery, fennel, potato, legume, resistant starch, ginger, onion, artichoke, olive, spinach, cabbage, brussels sprouts, carrot, leek, pepper, or mushroom extract. Vegetable extracts can be used as coloring agents as well as active ingredients.

Active agents may comprise one or more polyphenols. In some instances, the polyphenol comprises flavonoids, phenolic acids, polyphenolic amides, or other type of polyphenol. In some instances, flavonoids comprise quercetin, kaempferol, catechins, and anthocyanins. In some instances, phenolic acids comprise stilbenes or lignans. In some instances, polyphenolic amides comprise capsaicinoids or avenanthramides. In some instances, polyphenols comprise resveratrol, ellagic acid, curcumin, or lignans. In some instances, polyphenols comprise Anthocyanidins (e.g., cyanidin), Anthoxanthins, Flavones (e.g., apigenin), Flavanols (e.g., catechin), Flavanones (e.g., naringenin) Flavonols (e.g., quercetin and kaempferol), Flavans (e.g., leucoanthocyanidin), Isoflavones (e.g., daidzein), Isoflavanes (e.g., laxiflorane), Isoflavandiols, Isoflavenes (e.g., glabrene), Coumestans (e.g., wedelolactone) Pterocarpans (e.g., glyceollins), Stilbenoids (e.g., resveratrol), or Proanthocyanidins, Oligostilbenoids (e.g., α-viniferin).

Active agents may comprise compounds or mixtures obtained from botanical sources. In some instances an active agent comprises lecithinized botanical extracts. In some instances a lecithinized botanical extract comprises phytosomes. In some instances a lecithinized botanical extract comprises quercetin, grape seed, green tea, diindolylmethane (DIM), or curcuminoid.

A tea leaf extract can be a green tea extract or a black tea extract. A green tea extract can be obtained from Camellia spp such as Camellia sinensis. A green tea extract can include caffeine or be caffeine free (e.g., <1% w/w caffeine). A green tea extract can include at least 19% catechins (w/w: weight of catechin relative to weight of the green tea extract). Alternatively, a green tea extract can comprise at least 5%, 10%, 15%, 20%, or at least 25% catechins (w/w: weight of catechin relative to weight of the green tea extract). Catechins contemplated can be selected from the group consisting of: (−)-epigallocatechin; (+)-catechin; (−)-epicatechin; (−)-epigallocatechin 3-O-gallate; (+)-gallocatechin 3-O-gallate; (−)-epigallocatechin 3-O-(3′-O-methyl)-gallate; and (−)-epicatechin 3-O-gallate. In some instances, the green tea extract comprises at least 5%, 10%, 12%, 13%, 14%, 15%, or at least 20% (−)-epigallocatechin 3-O-gallate (EGCG) (w/w: weight of EGCG relative to weight of the green tea extract). Any of the green tea extracts herein can include one or more of hydrobenzoic acids; hydroxycinnamic acids; or flavones. A green tea extract can further comprises soy phospholipids. A matrix described herein comprises between about 2-25%, 2-20%, 5-15%, 5-10%, 6-9%, 10-20%, 26%, or 7-15% (w/w) of a green tea extract or about 5%, 6%, 7%, 8%, 9%, 10%, 12%, 15%, 16%, 17%, or 20% of a green tea extract (w/w) relative to the entire dissolvable matrix. In one example, a dissolvable matrix comprises about 15.69% green tea extract (w/w) relative to the entire dissolvable matrix

An active agent can be a sleep enhancer that improves sleep quality and/or quantity. The sleep enhancer can be a chamomile extract, L-theanine, melatonin, or a mixture thereof. The chamomile extract can be obtained from Matricaria recutita or Matricaria chamomilla. Chamomile extracts comprise at least 0.5%, 0.7%, 0.9%, 1%, 1.1%, 1.2%, 1.5%, or at least 2% apigenin (w/w: weight apigenin relative to weight of the chamomile extracts). When an active agent is a chamomile extract, a matrix described herein can comprise 1-15%, 1-20%, 5-25%, 10-20%, 10-25%, 15-25%, 5-10%, or 8-18% (w/w) chamomile extract relative to the entire dissolvable matrix. A matrix described herein can comprise about 5%, 7%, 9%, 10%, 12%, 15%, 16%, 17%, 20%, 22%, 25%, or about 30% (w/w) of a chamomile extract relative to the entire dissolvable matrix. When an active agent is L-theanine, a matrix described herein can comprise 10-15%, 10-20%, 5-35%, 10-40%, 10-45%, 15-50%, 25-40%, or 25-45% (w/w) of L-theanine relative to matrix weight. A matrix described herein can comprise about 5%, 10%, 15%, 20%, 25%, 30%, 35%, 40%, 45%, 50%, or about 55% (w/w) of L-theanine relative to matrix weight. When an active agent is melatonin, a matrix described herein can comprise 0.1-0.15%, 0.1-0.2%, 0.01-0.3%, 0.1-0.25%, 0.1-0.3%, 0.15-0.5%, 0.25-0.3%, or 0.08-0.2% (w/w) melatonin relative to matrix weight. A matrix described herein can comprise about 0.05%, 0.1%, 0.15%, 0.2%, 0.25%, 0.3%, 0.35%, 0.40%, or about 0.5% (w/w) melatonin relative to matrix weight. In one example, a dissolvable matrix comprises about 15.81% chamomile extracts (w/w), about 33.28% L-theanine (w/w), and about 0.17% melatonin (w/w), each relative to the entire dissolvable matrix.

An active agent can improve immunity. An active agent that improves immunity can be a lecithinized. The lecithinized product can be a quercetin lecithin complex. When an active agent is lecithinized, a matrix described herein can comprise 10-15%, 10-20%, 5-35%, 10-40%, 10-45%, 15-50%, 25-40%, or 25-45% (w/w) lecithin complex relative to the entire dissolvable matrix. A matrix described herein can comprise about 5%, 10%, 15%, 20%, 25%, 30%, 35%, 40%, 45%, 50%, or about 55% (w/w) lecithin complex relative to the entire dissolvable matrix. An active agent that improves immunity can additionally or alternatively be a vitamin or mineral, e.g., vitamin D3, ascorbic acid (i.e., vitamin C), a zinc salt, or zinc chelate (e.g., zinc picolinate). When an active agent is vitamin D3, a matrix described herein can comprise 1-5%, 1-10%, 1-3%, 2-8%, 3-10%, 2.5-4%, 1-4%, or 0.5-4% (w/w) vitamin D3 relative to matrix weight. A matrix described herein can comprise about 0.5%, 1%, 1.5%, 2.0%, 2.5%, 3.0%, 3.5%, 4.0%, 4.5%, 5.0%, or about 5.5% (w/w) vitamin D3 relative to the entire dissolvable matrix. When an active agent is ascorbic acid, a matrix described herein can comprise 10-15%, 10-20%, 5-35%, 10-40%, 10-45%, 15-50%, 25-40%, or 25-45% (w/w) of ascorbic acid or about 5%, 10%, 15%, 20%, 25%, 30%, 35%, 40%, 45%, 50%, or about 55% (w/w) of ascorbic acid, each relative to the entire dissolvable matrix. When an active agent is a zinc salt, a matrix described herein can comprise about 5-15%, 5-20%, 5-35%, 10-40%, 10-45%, 15-50%, 25-40%, or 25-45% (w/w) of zinc salt or about 1%, 2%, 5%, 10%, 15%, 20%, 25%, 30%, 35%, 40%, or about 55% (w/w) zinc salt relative to matrix weight. In some embodiments, the zinc salt is a zinc chelate. In some embodiments, the zinc salt is zinc picolinate. In one example, a dissolvable matrix comprises about 29.83% quercetin lecithin complex (w/w), about 3.58% vitamin D3 (w/w), about 13.72% ascorbic acid (w/w), and about 8.95% zinc picolinate (w/w), each relative to the entire dissolvable matrix.

Provided herein are active agents for generating dissolvable matrices, described in Table 2

TABLE 2

| Active Agent | Example Product | Manufacturer/Source |
|---|---|---|
| bacteriophage/prebiotic | PreforPro | Deerland |
| blueberry extract | AuroraBlue | Denali Biotech |
| green tea extract | Greenselect ® Phytosome | Indena |
| pomegranate extract | Pomma+ | Hawkins, Inc |

Other active agents, products, and sources consistent with the disclosure herein are also contemplated.

Active agents may include supplements and the supplements may include those suitable for nutrition, flavor enhancement, and/or medicinal purposes that can be ingested. Nutritional supplements can include a vitamin, a mineral, a protein, a probiotic, a fiber, an amino acid, and other dietary supplements. For example, vitamins in some instances include any suitable vitamin that can be ingested, such as vitamin A, B, C, D, E, B12, and the like found in a typical over the counter multivitamin. Minerals in some instances include iron, magnesium, potassium, and the like found in a typical over the counter multivitamin/multimineral. A protein in some instances includes whey protein or a plant-based protein. In some instances, the active and inactive ingredients include pharmaceuticals, such as acetylsalicylic acid, acetaminophen, ibuprofen, etc., as well as beverage and food items.

An active agent or mixture of two or more active agents described herein may comprise a lipophilicity value or be designated lipophilic. Such a classification in some instances is used to choose optimum conditions for generating a dissolvable matrix. In some instances, the lipophilicity is measured by a log P value, wherein higher values indicate more lipophilic character. In some instances, lipophilicity is compared to a threshold value to determine if the agent or mixture is lipophilic (e.g., if a threshold is 1.5 and the agent is 2, the agent is lipophilic). In some instances, the lipophilicity threshold is about −1.5, −1.25, −1, −0.5, −0.25, 0.25, 0.5, 0.75, 1, 1.25, 1.5, 1.75, 2, 2.25, 2.5, 2.75, 3, 3.5, 4, 4.5, 5, 6, 7, 8, 9 or about 10. In some instances, the lipophilicity threshold is at least −1.5, −1.25, −1, −0.5, −0.25, 0.25, 0.5, 0.75, 1, 1.25, 1.5, 1.75, 2, 2.25, 2.5, 2.75, 3, 3.5, 4, 4.5, 5, 6, 7, 8, 9 or at least 10. In some instances, the lipophilicity threshold is −1.5-10, −1.5-5, −1.5-5, −0.01-10, 0.01-7, 0.01-5, 0.01-2, 0.05-5, 0.1-10, 0.2-10, 0.5-10, 1-10, 1.5-10. 2-10, or 5-15.

### Coloring

A dissolvable matrix can have one or more colors added. A natural colorant, such as turmeric, beet root, or another colorant, is used. Colorants may be chosen to resist photobleaching or color change during sunlight exposure or storage. In some instances, an artificial colorant added, such a food-grade coloring.

### Flavorings

A dissolvable matrix may comprise one or more excipients to create a flavor. A dissolvable matrix may comprise a sweetener. Sweeteners include but are not limited to xylitol, sugar, dextrose, acesulfame potassium, aspartame, neotame, saccharin, sucralose, or Stevia extract. Natural or artificial sweeteners may be used. In some instances, a flavoring comprises an extract from a fruit, such as lemon (e.g., Meyer lemon), orange, cherry (e.g., acerola), raspberry, watermelon, apple, pomegranate, or other fruit. Extracts may be blended to produce additional flavors. Citric acid may be used as a flavoring excipient, e.g., to provide acidity.

### Matrix Shape/Size/Weight/Packaging

A solid dissolvable matrix can be in the form of a rectangular or square strip, sheets, a cube, a sphere, a disk, oval, star, snowflake, decorative design, recognizable shape (e.g., animal shape, logo, icon, or TV/movie/comic character) and the like. The dissolvable matrix can vary in dimensions. Such variation in size may be dependent on application. In some instances, an individual matrix may range in length or diameter from about 1 mmxl mm to about 12 inches by 12 inches; these larger lengths or diameters are possible, for example, such as when the dissolvable matrix is formed (i.e., printed) as a sheet for large-scale production or when a large sheet is cut into individual matrices. Larger sizes for printed compositions may be utilized in large tea makers or large industrial coffee makers. In some instances, the matrix is substantially two dimensional (sheet, disk, rectangle, square, donut, strip, or other shape). In some instances, the matrix is substantially three dimensional (cylinder, sphere, cube, prism, pyramid, torus). In some instances, a two dimensional matrix comprises a smallest linear dimension that is about 2, 5, 10, 20, 30, 40, 50, 60, 70, 80, 90, 100, 125, 150, 175, or about 200 times smaller than the largest linear dimension. In some instances, a two dimensional matrix comprises a smallest linear dimension which is about 2-200, 2-150, 2-125, 2-100, 2-50, 2-25, 5-500, 5-250, 5-150, 10-100, 10-200, 10-300, 25-400, 25-300, 25-250, 25-100, 50-100, 50-200, 50-300, 50-500 100-250, 100-500, 100-500, or 250-500 times smaller than the largest linear dimension.

In some instances, a dissolvable matrix has a longest cross-sectional length of 0.1-0.2, 0.2-0.3, 0.3-0.4, 0.5-4, 0.5-3, 0.5-3, 1-3, 1-2, 1.5-3.5 or 1.5-2.5 inches. In some instances, a dissolvable matrix has a longest cross-sectional length of about 0.25, 0.5, 0.75, 1, 1.25, 1.5, 1.75, 2, 2.25, 2.5, 2.75, or about 3 inches. In some instances, a dissolvable matrix has a longest cross-sectional length of no more than 0.25, 0.5, 0.75, 1, 1.25, 1.5, 1.75, 2, 2.25, 2.5, 2.75, or no more than 3 inches. The dissolvable matrix may have the size and shape of an 8½″ by 11″ cover sheet. In some instances the dissolvable matrix comprises a largest cross-sectional area of about 0.5, 1, 1.25, 1.5, 2.0, 2.25, 2.5, 3.75, 4, 4.25, 4.5, 4.75, 5, 5.5, 6, 6.5, or about 7 square inches. In some instances the dissolvable matrix comprises a largest cross-sectional area of no more than 0.5, 1, 1.25, 1.5, 2.0, 2.25, 2.5, 3.75, 4, 4.25, 4.5, 4.75, 5, 5.5, 6, 6.5, or no more than 7 square inches. In some instances the dissolvable matrix comprises a largest cross-sectional area of at least 0.5, 1, 1.25, 1.5, 2.0, 2.25, 2.5, 3.75, 4, 4.25, 4.5, 4.75, 5, 5.5, 6, 6.5, or at least 7 square inches. In some instances the dissolvable matrix comprises a largest cross-sectional area of 0.2-10, 0.5-10, 1-10, 2-10, 0.5-7, 1-7, 2-5, 2-7, 2-12, 3-5, 3-10, or 5-10 square inches.

A dissolvable matrix may range in thickness from about 1 microns to about 50 mm, or greater than 15 mm. In some instances, a disc's thickness is measured as an average thickness. A dissolvable matrix may have a thickness of 50-500, 100-1000, 100-500, 200-700, 300-700, 400-500, or 400-1000 microns. In some instances, the thickness is about 1, 10, 50, 100, 150, 200, 250, 300, 350, 400, 450, 500, 600, 700, 800, 900, 1000, 1100, 1200, 1300, 1400, 1500, or about 2000 microns. In some instances, the thickness is no more than 1, 10, 50, 100, 150, 200, 250, 300, 350, 400, 450, 500, 600, 700, 800, 900, 1000, 1100, 1200, 1300, 1400, 1500, or no more than 2000 microns. In some instances, the thickness is 250 to 1000 microns. In some instances, the thickness is 150 to 1500 microns. In some instances, the thickness is between 50 to 500 microns. In some instances, the thickness is between 50-500, 25-1000, 25-500, 25-250, 25-100, 50-250, 75-750, 100-250, 100-500, 100-1000, or between 45-750 microns.

The solid dissolvable matrix may comprise any shape. In some instances, the solid dissolvable matrix is in a disc shape. The disc may have a diameter of 0.5-4, 0.5-3, 0.5-3, 1-3, 1-2, 1.5-3.5 or 1.5-2.5 inches and has a thickness of 100-2000, 100-5000, 100-1000, 100-500, 200-700, 300-700, 400-500, 400-1000, 500-2000, 750-2000, 800-2000, or 1200-5000 microns. In some instances, the disc has a diameter of 0.5-4, 0.5-3, 0.5-3, 1-3, 1-2, 1.5-3.5 or 1.5-2.5 inches. In some instances, the disc has a diameter of about 0.25, 0.5, 0.75, 1, 1.25, 1.5, 1.75, 2, 2.25, 2.5, 2.75, or about 3 inches. In some instances, the disc has a diameter of no more than 0.25, 0.5, 0.75, 1, 1.25, 1.5, 1.75, 2, 2.25, 2.5, 2.75, or no more than 3 inches. In some instances, the disc has a thickness of 100-2000, 100-5000, 100-1000, 100-500, 200-700, 300-700, 400-500, 400-1000, 500-2000, 750-2000, 800-2000, or 1200-5000 microns. In some instances, the disc has a thickness of about 100, 150, 200, 250, 300, 350, 400, 450, 500, or about 700 microns.

Dissolvable matrices may comprise a variety of weights. In some instances, the dissolvable matrix is at least 100, 200, 500, 800, 1000, 2000, or 5000 mg (in total, i.e., including active agents and excipients). In some instances, the dissolvable matrix is about 500-800, 100-800, 50-200, 100-500, 200-800, 500-1000, or 500-1500 mg. In some instances, the dissolvable matrix is about 1 microgram or more, e.g., 1 μg, 2 μg, 3 μg, 4 μg, 5 μg, 6 μg, 7 μg, 8 μg, 9 μg, 10 μg, 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, 900 μg, 1000 μg or more. In some instances, the dissolvable matrix is about 1 milligram or more, e.g., 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, or 500 mg.

The size and shape of each matrix is in some instances tailored depending upon its intended use, its packaging, and/or its shipping method. For instance, the matrix is printed in a rectangular shape, such that the matrix is rectangular and capable of being positioned within a box for flat shipping. In another instance, the matrix is printed in a circular shape, such that the matrix is circular and capable of packaged in a tube. Circular dissolvable matrices are particularly suitable for being added to beverages in a glass or another circular container for dissolution.

In some instances, a dissolvable matrix described herein is placed in packaging for transport or commercial sale. In some instances, the packaging is substantially air and water tight. In some instances, packaging further comprises an ingredient label. In some instances, packaging further comprises instructions for use.

The dissolvable matrix may be arranged in various dispensing configurations. For example, a dispensing configuration comprises continuous tape with or without perforations for tearing. In some instances, the continuous tape arrangement of the dissolvable matrix is placed in a tape dispenser type device, where a portion of the strip may be torn off with the assistance of a cutting blade. In the continuous tape arrangement, a support substrate, such as release paper in some instances supports and or encloses the dissolvable matrix. In another exemplary embodiment, the dissolvable matrix is printed on a release paper in the form of dots, small particles, granules, or the like. The dissolvable matrix is then be removed from the release paper. In instances where the dissolvable matrix is formed on a printed support substrate, no release paper may be required. Rather, the dissolvable matrix may be stored in a dispenser such as a dispenser with openings similar manner to that of a salt shaker. Other dispensing configurations include but are not limited to stacking the dissolvable matrix on top of each, such as similar to Pez® candy from a Pez® candy dispenser, or packaging the dissolvable matrix in a pouch or sealed packaging, similar to an individual bandage. Packaging for dispensing in some instances is printed and/or individualized, for example, with a person's name, companies name, or company logo.

### Uses

Provided herein are methods comprising promoting or maintaining a physical status or condition of a subject. In some instances, methods comprise administering a dissolvable composition described herein. In some instances, a dissolvable composition is administered wherein the dissolvable composition comprises one or more active agents. In some instances, the active agent comprises a nutraceutical. In some instances, a method for promoting a condition of a subject comprises administering a dissolvable composition described herein. In some instances, a method of maintaining a physical status or a condition of a subject comprises administering a dissolvable composition described herein. In some instances, the condition comprises youthfulness appearance (healthy skin and cell membranes, softness, wrinkle reduction, regulation of oil production, regulation of hydration, reduction of hyperkeratinization of hair follicles, reduction of premature aging, reduction of acne, and reduction or prevention of sun damage), reduced depression or anxiety (or associated symptoms such as sadness, lethargy, or general loss of interest in life), eye health (healthy visual acuity, night vision, and health of specific eye structures, such as retina or macula, tear production, or other eye-related health issue), cognitive function (intelligence, improved communication and social skills, decreasing hyperactivity, impulsiveness, restlessness, aggression, reduction in mood swings, reducing age-related mental decline, or related diseases), digestive health (microbiome composition, regularity, reduction in bloating) cardiovascular health (reduction in triglycerides, blood pressure, blood clots, arterial plaques, inflammation, regulation of cholesterol levels, such as by increasing levels of high-density lipids), healthy immune system (lowering the risk or symptoms of diseases such as diabetes, multiple sclerosis, lupus, rheumatoid arthritis, ulcerative colitis, Crohn's disease, or psoriasis), supporting a reduced risk of cancer (colon cancer, prostate cancer, breast cancer, or other cancer), respiratory health (asthma or associated symptoms such as coughing, shortness of breath, or wheezing), liver health (fat in the liver, or conditions such as non-alcoholic fatty liver disease), healthy bone and joint health (lowering the risk of osteoporosis by increasing calcium levels, and/or reducing arthritis and increasing grip strength), pain reduction (reduce lower abdominal or pelvic pain, such as menstrual pain), sleep health (length of sleep, quality of sleep, regulation of sleep cycles, regulation of melatonin), urinary health, regulation of metabolic functions, and healthy pregnancy (including prenatal development). In some instances, the condition comprises healthy sleep. In some instances, the condition comprises performance. In some instances, the condition comprises digestive health. In some instances, the condition comprises a healthy immune system. The matrices herein can be used to promote or maintain a healthy brain, healthy mood, cardiovascular health, blood sugar, glucose metabolism, weight management or optimal weight, healthy aging, reduction of oxidative stress, a healthy inflammatory response (e.g., in the central nervous system), and/or reduction of lipid accumulation in fat cells. Also, they can be used to promote higher quality and/or quantity sleep. In some instances, a method comprises administering a dissolvable matrix herein for promoting a condition of a subject, wherein the condition comprises a healthy brain, healthy mood, cardiovascular health, blood sugar, glucose metabolism, weight management or optimal weight, healthy aging, reduction of oxidative stress, a healthy inflammatory response (e.g., in the central nervous system), and/or reduction of lipid accumulation in fat cells. Also, they can be used to promote higher quality and/or quantity sleep. In some instances, a method comprises administering a dissolvable matrix herein for maintaining a condition of a subject, wherein the condition comprises a healthy brain, healthy mood, cardiovascular health, blood sugar, glucose metabolism, weight management or optimal weight, healthy aging, reduction of oxidative stress, a healthy inflammatory response (e.g., in the central nervous system), and/or reduction of lipid accumulation in fat cells. Also, dissolvable matrices can be used to promote higher quality and/or quantity sleep.

The dissolvable matrices may be used sublingually, orally, added to a food or beverage item, etc., depending upon their configuration. For instance, dissolvable matrices which are configured to dissolve/disperse easily may be used sublingually, whereas a dissolvable matrix which is configured to dissolve may be used within a beverage item. The foregoing examples are in no way limiting, as slow dissolving/dispersing dissolvable matrices may be used in a beverage and a fast dissolving/dispersing dissolvable matrix may be used orally.

The beverage in some instances includes but is not limited to juice, water, tea, milk, coffee, fermented beverages (beer, wine, kombucha), soda, or other solvent.

The dissolvable matrix may be used in various applications that may benefit from a supplement. For example, the dissolvable matrix is added to a food or beverage, such as a tea bag, a coffee pod, and the like. In one example, as the tea in a tea bag and/or coffee in a coffee pod is made using water, a supplement from the printed composition within the tea bag and/or coffee pod, is released in the tea and/or coffee.

### Methods of Manufacture

A dissolvable matrix disclosed herein can be produced using one or more of the following manufacturing methodologies.

A first method for making a dissolvable matrix comprises 1) mixing one or more active agents and at least one excipient in a solvent to produce a mixture, 2) printing the mixture, and 3) curing the mixture. The method can further comprise the step of shaping the mixture.

Typically, a plurality of active agents and excipients are combined with a liquid (e.g., purified water) to create a homogenous liquid composition that is capable of being printed. Ingredients (e.g., active agents and excipients) are mixed with solvents (e.g., purified water) at a suitable temperature. During the mixing process, when air is incorporated into the liquid composition, this process is referred to as blowing.

The liquid composition is mixed to achieve a desired initial viscosity. An initial viscosity is measured in cP (millipascal-second). An initial viscosity may be 1000-25000, 2000-25000, 5000-25000, 8000-25000, 1000-12000, 4000-15000 5000-20000, or 2000-15000 cP. In some instances, a mixture provided herein has an initial viscosity of 5000-13000 cP, 7000-20000 cP, or 3000-10000 cP.

Printing provides a product that may be roughly the desired shape and size, albeit larger and/or heavier than the final product due to the presence of a liquid component that remains to be omitted, e.g., dried.

The dissolvable matrix can be printed using any number of printing techniques. The matrix may be made by screening printing, rotary screen printing, flexography, offset gravure, ink jet, bubble jet, dry toner, ribbon transfer, powder coating, spray coating, roll coating, reverse roll coating, slot die coating, hot and/or cold laminating, knife coating, sintering, padding, or curtain coating, and the like. In this regard, printing techniques are understood to cover coating techniques. In some instances, the matrix is made using printing.

Curing is notable in that a dissolvable acquires its final, solid shape after curing. In other words, during curing volatile and liquid components of the printed mixture are evaporated, dried, or the like, leaving a shelf-stable, solid dissolvable matrix. Curing can occur at room temperature up to about 95° C.

Curing steps may be performed at a specific temperature or range of temperatures, for a period of time. In some instances, a curing step is performed for a time until the dissolvable matrix reaches a desired water content (e.g., dried), e.g., a water content of less than 10%, 8%, 7%, 6%, 5%, 4%, 3%, or less than 2% (w/w). A matrix may be cured at a temperature of 50-90, 50-70, 60-80, 65-90, 65-80, 70-80, 70-85, 60-70, or 65-75° C. A matrix may be dried for at least 1, 2, 5, 8, 10, or more than 10 hours, e.g., 0.5-5, 1-5, 2-5, 2-10, 3-15, 5-24, or 8-16 hours.

In some cases, curing ends when a dissolvable matrix has achieved a moisture content of less than 4%. The moisture content may be measured by water balance.

Stencils of different sizes and shapes may be used to produce dissolvable matrices of a desired size and shape. In some instances, the stencil gauge is 10-25, 12-15, 12-22, 13-18, 15-20, or 14-20. In some instances, the stencil gauge is 14. In some instances, the stencil gauge is 20. In some instances, the stencil gauge is 16.

In some instances, shaping comprises use of a blade and/or other cutting instrument to achieve a desired shape and size

In some instances, decorative designs or other such indicia, such as words, pictures, etc., may be printed onto the dissolvable matrix. In this regard, indicia may include the identification codes, such as spatial codes, QR codes, bar codes, identification numbers, or other such indicia which can be used to identifying, track, and/or provide information. These indicia and decorative designs may be ink-jet or flexographic printed directly onto the dissolvable matrix. The ink used may be culturally- and/or dietary-acceptable inks, e.g., vegetarian, vegan, halal, and kosher. Any other printing technique may be used, such as screening printing, rotary screen printing, flexography, offset gravure, ink jet, bubble jet, dry toner, ribbon transfer, powder coating, spray coating, roll coating, reverse roll coating, slot die coating, hot and/or cold laminating, knife coating, sintering, padding, curtain coating, and the like. In this regard, printing techniques are understood to cover coating techniques. The ink may be aqueous or solvent based. The ink may be ultraviolet (UV) curable, electron beam (EB) curable, thermally curable, cold curable, ambient catalyzed, ambient crosslinked, and the like. The ink may be edible and/or dissolvable based on the desired application.

### Illustrative Dissolvable Matrices

FIG. 1 illustrates a method for determining matrix classes based on an active agent or combinations of active agents. In some instances, each matrix type (e.g., 1, 1a, 2, 2a, as disclosed herein) is associated, at least, with a specific set and/or concentration of excipients.

A class 1 dissolvable matrix, as used herein, comprises one or more active agents in which the active agent or combination of active agents is water soluble and not hydroscopic. Class 1 matrices comprise active agents, pore-creating excipients, pore-size modifying excipients, and emulsifiers. An illustrative class 1 matrix comprises blueberry powder, green tea extract, pomegranate powder, and a bacteriophage component. Another illustrative class 1 matrix comprises one or more B vitamins, e.g., riboflavin-5-phosphate, methylcobalamin, L-methyltetrahydrofolate (L-MTHF) calcium salt, pyridoxal-5-phosphate (P5P), and thiamine HCl. In class 1 matrices, the pore-creating excipient is cellulose powder and/or quillaja extract.

A class 1a dissolvable matrix, as used herein, comprises one or more active agents in which the active agent or combination of active agents is water soluble and is hydroscopic. Class 1a matrices comprise active agents, pore-creating excipients, pore-size modifying excipients, and mineral ions/mineral ion donors, and, optionally, hygroscopicity modifiers. One illustrative class 1a matrix comprises blueberry powder, green tea extract, pomegranate powder, and a bacteriophage component as active agents. Other illustrative class 1a matrices comprise Other illustrative class 1a matrices comprise mango leaf extract, methylcobalamin, L-methyltetrahydrofolate methyltetrahydrofolate Ca, and Pyridoxal-5-Phosphate as active agents. The pore-creating excipients are cellulose powder and quillaja extract. The hygroscopicity modifier, when present, is MCT oil powder. The pore-size modifying excipient is microcrystalline cellulose.

A class 2 dissolvable matrix, as used herein, comprises one or more active agents in which the active agent or combination of active agents is water insoluble and is lipophilic. Class 2 matrices comprise active agents, pore-creating excipients, pore-size modifying excipients, and emulsifiers. One illustrative class 2 matrix comprises chamomile extract, L-Theanine, and melatonin as active agents. The pore-creating excipients are cellulose powder and quillaja extract. The pore-size modifying excipient is oat fiber. The emulsifier is carboxymethyl cellulose gum (CMC gum).

A class 2a dissolvable matrix, as used herein, comprises one or more active agents in which the active agent or combination of active agents is water insoluble and is not lipophilic, and one of the active agents comprises lecithin. Class 2a matrices comprise active agents (at least one of which is a lecithin), pore-creating excipients, pore-size modifying excipients, and mineral ions/mineral ion donors. One illustrative class 2a matrix comprises a quercetin lecithin complex, vitamin D3, ascorbic acid (vitamin C), a zinc salt (e.g., zinc picolinate), or zinc chelate. The pore-creating excipients are cellulose powder and quillaja extract. The pore-size modifying excipient is tapioca starch. The mineral ions/mineral ion donors is calcium carbonate.

### Numbered Embodiments

Provided herein are numbered embodiments 1-127: 1. A dissolvable matrix comprising: at least 30% (w/w, relative to the dissolvable matrix) of one or more active agents and at least one excipient, wherein the at least one excipient is configured for pore creation and creates structure in the matrix; wherein the dissolvable matrix comprises 1-70% void volume (v/v, relative to the dissolvable matrix), and wherein the matrix is configured to dissolve in an aqueous solution. 2. The dissolvable matrix of embodiment 1, wherein the at least one excipient is powdered cellulose or quillaja extract. 3. The dissolvable matrix of embodiment 2, wherein the dissolvable matrix comprises powdered cellulose and quillaja extract. 4. The dissolvable matrix of embodiment 2 or 3, wherein the powdered cellulose is 10-35% (w/w) relative to the dissolvable matrix. 5. The dissolvable matrix of any one of embodiments 2 to 4, wherein the quillaja extract is 0.5-10% (w/w) relative to the dissolvable matrix. 6. The dissolvable matrix of any one of embodiments 1 to 5, wherein the dissolvable matrix comprises a plurality of pores having an average largest cross-sectional area of about 0.1-10 microns. 7. The dissolvable matrix of any one of embodiments 1 to 6, wherein the dissolvable matrix comprises at least 40% (w/w) of the one or more one active agents relative to the dissolvable matrix. 8. The dissolvable matrix of any one of embodiments 1 to 7, wherein the dissolvable matrix comprises about 50-90% (w/w) of the one or more active agents relative to the dissolvable matrix. 9. The dissolvable matrix of any one of embodiments 1 to 8, wherein the dissolvable matrix comprises the shape of a cylinder or tablet. 10. The dissolvable matrix of embodiment 9, wherein the cylinder is no more than 500 microns thick and no more than two inches in diameter. 11. The dissolvable matrix of embodiment 9 or 10, wherein the cylinder is 400-500 microns thick and two inches in diameter. 12. The dissolvable matrix of any one of embodiments 1 to 11, wherein the dissolvable matrix has a surface area of at least 8000 mm2. 13. The dissolvable matrix of any one of embodiments 1 to 12, wherein the dissolvable matrix comprises a shape of an animal. 14. The dissolvable matrix of any one of embodiments 1 to 12, wherein the dissolvable matrix comprises no more than 8% water (w/w) relative to the dissolvable matrix. 15. The dissolvable matrix of any one of embodiments 1 to 12, wherein the dissolvable matrix comprises no more than 6% water (w/w) relative to the dissolvable matrix. 16. The dissolvable matrix of any one of embodiments 1 to 12, wherein the dissolvable matrix comprises no more than 4% water (w/w) relative to the dissolvable matrix. 17. The dissolvable matrix of any one of embodiments 1 to 16, wherein the aqueous solution is juice, water, tea, milk, coffee, a fermented beverage (beer, wine, kombucha), or soda. 18. The dissolvable matrix of any one of embodiments 1 to 17, wherein the dissolvable matrix is configured to dissolve in water having a temperature of no more than 30° C. in less than 10 seconds. 19. The dissolvable matrix of any one of embodiments 1 to 17, wherein the dissolvable matrix is configured to dissolve in water having a temperature of no more than 20° C. in less than 60 seconds. 20. The dissolvable matrix of any one of embodiments 1 to 17, wherein the dissolvable matrix is configured to dissolve in water having a temperature of no more than 10° C. in less than 60 seconds. 21. The dissolvable matrix of any one of embodiments 1 to 17, wherein the dissolvable matrix is configured to dissolve in water having a temperature of no more than 5° C. in less than 60 seconds. 22. The dissolvable matrix of any one of embodiments 1 to 17, wherein the dissolvable matrix is configured to dissolve in water having a temperature about 0° C. in less than 60 seconds. 23. The dissolvable matrix of embodiment any one of embodiments 1 to 17, wherein the dissolvable matrix is configured to dissolve in no more than 8 oz of water having a temperature of no more than 20° C. in less than 60 seconds. 24. The dissolvable matrix of embodiment any one of embodiments 1 to 17, wherein the dissolvable matrix is configured to dissolve in no more than 8 oz of water having a temperature of no more than 20° C. in less than 30 seconds. 25. The dissolvable matrix of embodiment any one of embodiments 1 to 24, wherein the dissolvable matrix is configured to dissolve in an aqueous solution having a pH of 2-10. 26. The dissolvable matrix of any one of embodiments 1 to 25, wherein the void volume is at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, or at least about 70% (v/v) relative to the dissolvable matrix. 27. The dissolvable matrix of any one of embodiments 1 to 25, wherein the void volume is about 1%, about 5%, about 10%, about 15%, about 20%, about 25%, about 30%, about 35%, about 40%, about 45%, about 50%, about 55%, about 60%, about 65%, or about 70% (v/v) relative to the dissolvable matrix. 28. The dissolvable matrix of any one of embodiments 1 to 27, wherein dissolvable matrix comprises a balance of pore size and pore distribution that provides desirable tensile strength, dissolution speed, and moisture transfer rates. 29. The dissolvable matrix of any one of embodiments 1 to 28, wherein the dissolvable matrix is shelf stable. 30. A dissolvable matrix comprising: at least 30% (w/w) of one or more active agents and at least one excipient, wherein the at least one excipient is configured for pore creation and creates structure in the matrix; and wherein the matrix is configured to dissolve in an aqueous solution. 31. The dissolvable matrix of embodiment 30, wherein the at least one excipient is powdered cellulose or quillaja extract. 32. The dissolvable matrix of embodiment 30, wherein the at least one excipient comprises powdered cellulose and quillaja extract. 33. The dissolvable matrix of embodiment 31 to 32, wherein the powdered cellulose is 10-35% (w/w). 34. The dissolvable matrix of embodiment 31 to 32, wherein the quillaja extract is 0.5-10% (w/w). 35. The dissolvable matrix of embodiment any one of embodiments 30 to 34, wherein the at least one excipient is configured for pore size/distribution modification and/or emulsifier stabilization. 36. The dissolvable matrix of embodiment 35, wherein the excipient has a D50 of 50-150 microns. 37. The dissolvable matrix of embodiment 30 to 26, wherein the excipient is microcrystalline cellulose. 38. The dissolvable matrix of any one of embodiments 30 to 37, wherein the excipient is tapioca starch, microcrystalline cellulose, or Oat fiber. 39. The dissolvable matrix of any one of embodiments 30 to 38, wherein the excipient is tapioca starch or Oat fiber. 40. The dissolvable matrix of any one of embodiments 37 to 38, wherein the microcrystalline cellulose is 5-15% (w/w). 41. The dissolvable matrix of any one of embodiments 30 to 40, wherein the at least one excipient is an emulsifier. 42. The dissolvable matrix of embodiment 41, wherein the emulsifier comprises CMC gum. 43. The dissolvable matrix of any one of embodiments 30 to 42, wherein the at least one excipient is a hygroscopicity modifier. 44. The dissolvable matrix of embodiment 43, wherein the hygroscopicity modifier comprises medium chain triglycerides. 45. The dissolvable matrix of embodiment 44, wherein the medium chain triglycerides are 1-5% (w/w). 46. The dissolvable matrix of any one of embodiments 30 to 45, wherein the at least one excipient is a mineral ion donor. 47. The dissolvable matrix of embodiment 46, wherein the mineral ion donor is a calcium salt, e.g., calcium carbonate. 48. The dissolvable matrix of any one of embodiments 46 to 48, wherein the mineral ion donor is 1-10% (w/w). 49. The dissolvable matrix of any one of embodiments 30 to 49, wherein the at least one excipient is a pullulan. 50. The dissolvable matrix of embodiment 50, wherein the pullulan is 1-5% (w/w). 51. The dissolvable matrix of any one of embodiments 30 to 51, wherein the at least one excipient is a glycerin. 52. The dissolvable matrix of embodiment 52, wherein the glycerin is 2-15% (w/w). 53. The dissolvable matrix of any one of embodiments 30 to 53, wherein the at least one excipient comprises plant fibers, oils, gums, or collagen. 54. The dissolvable matrix of any one of embodiments 30 to 53, wherein dissolvable matrix comprises a balance of pore size and pore distribution that provides desirable tensile strength, dissolution speed, and moisture transfer rates and is shelf stable. 55. A dissolvable matrix comprising: at least 30% (w/w) of one or more active agents, wherein one or more of the active agents is a prebiotic; and at least one excipient, wherein the matrix is configured to dissolve in an aqueous solution. 56. The dissolvable matrix of embodiment 55, wherein the prebiotic is a bacteriophage component or a polyphenol component. 57. The dissolvable matrix of embodiment 56, wherein the matrix comprises at least one bacteriophage component and at least one polyphenol component. 58. The dissolvable matrix of any one of embodiments 55 to 57, wherein the prebiotic is a bacteriophage component. 59. The dissolvable matrix of embodiment 58, wherein the bacteriophage component comprises one or more lytic bacteriophages. 60. The dissolvable matrix of embodiment 59, wherein the one or more lytic bacteriophages are of the Siphoviridae or Myoviridae family. 61. The dissolvable matrix of embodiment 59, wherein the one or more lytic bacteriophages are selected from LH01-Myoviridae, LL5-Siphoviridae, T4D-Myoviridae, or LL12-Myoviridae. 62. The dissolvable matrix of embodiment 58, wherein the bacteriophage component accelerates the growth of one or more of B. bifidum; B breve; B. animalis subsp. lactis; B. longum; L. acidophilus; L. paracasei; L. plantarum; L. rhamnosus; or B. subtilis. 63. The dissolvable matrix of embodiment 58, wherein the bacteriophage component supports increases in the concentration of butyrate-producing Eubacteria, decreases the concentration of Clostridium perfringens, or decreases interleukin 4 (IL-4) cytokine. 64. The dissolvable matrix of any one of embodiments 58 to 63, wherein the bacteriophage component is 1-5% (w/w). 65. The dissolvable matrix of any one of embodiments 58 to 64, wherein the bacteriophage component is about 3% (w/w). 66. The dissolvable matrix of embodiment 56 or 57, wherein the polyphenol component comprises a fruit extract, vegetable extract, or tea leaf extract. 67. The dissolvable matrix of embodiment 56 or 57, wherein the polyphenol component comprises one or more of blueberry extract, green tea extract, and pomegranate extract. 68. The dissolvable matrix of embodiment 67, wherein the polyphenol component is blueberry extract. 69. The dissolvable matrix of embodiment 68, wherein the blueberry extract is derived from Vaccinium spp. 70. The dissolvable matrix of embodiment 69, wherein the Vaccinium spp. is one or more of Vaccinium alaskaense How; Vaccinium ovaliforium Sm; Vaccinium membranaceum L.; Vaccinium uliginosum L.; or Vaccinium cespitosum Mich X. 71. The dissolvable matrix of embodiment 69, wherein the blueberry extract is configured to promote healthy brain and mood, cardiovascular health, blood sugar maintenance, optimal weight, and/or healthy aging. 72. The dissolvable matrix of embodiment 69, wherein the blueberry extract supports reducing oxidative stress and a healthy response to inflammation in the central nervous system, reduced lipid accumulation in fat cells, and maintenance of blood sugar levels already within a healthy range. 73. The dissolvable matrix of any one of embodiments 69 to 73, wherein the blueberry extract is 10-20% (w/w). 74. The dissolvable matrix of any one of embodiments 69 to 73, wherein the blueberry extract is about 15% (w/w). 75. The dissolvable matrix of embodiment 66, wherein the polyphenol component is green tea extract. 76. The dissolvable matrix of embodiment 75, wherein the green tea extract is obtained from Camellia spp. 77. The dissolvable matrix of embodiment 76, wherein the green tea extract is obtained from Camellia sinensis. 78. The dissolvable matrix of any one of embodiments 75 to 77, wherein the green tea extract comprises at least 19% catechins (w/w). 79. The dissolvable matrix of embodiment 78, wherein the catechins are selected from (−)-epigallocatechin; (+)-catechin; (−)-epicatechin; (−)-epigallocatechin 3-O-gallate; (+)-gallocatechin 3-O-gallate; (−)-epigallocatechin 3-O-(3′-O-methyl)-gallate; and (−)-epicatechin 3-O-gallate. 80. The dissolvable matrix of embodiment 79, wherein the green tea extract comprises at least 13% (−)-epigallocatechin 3-O-gallate (EGCG) (w/w). 81. The dissolvable matrix of any one of embodiments 75 to 77, wherein the green tea extract comprises one or more of hydrobenzoic acids; hydroxycinnamic acids; or flavones. 82. The dissolvable matrix of any one of embodiments 75 to 81, wherein the green tea extract further comprises soy phospholipids. 83. The dissolvable matrix of any one of embodiments 75 to 81, wherein the green tea extract supports weight management, cardiovascular health, glucose metabolism, and/or a healthy inflammatory response. 84. The dissolvable matrix of any one of embodiments 75 to 83, wherein the green tea extract is 10-20% (w/w). 85. The dissolvable matrix of any one of embodiments 75 to 84, wherein the green tea extract is about 16% (w/w). 86. The dissolvable matrix of embodiment 67, wherein the polyphenol component is pomegranate extract. 87. The dissolvable matrix of embodiment 86, wherein the pomegranate extract comprises ellagitannins or punicalagins. 88. The dissolvable matrix of embodiment 86, wherein the pomegranate extract is obtained from Punica spp. 89. The dissolvable matrix of embodiment 88, wherein the pomegranate extract is obtained from Punica granatum. 90. The dissolvable matrix of embodiment 86 to 88, wherein the pomegranate extract supports reduction of oxidative damage, cardiovascular health, and/or a healthy immune system. 91. The dissolvable matrix of any one of embodiments 86 to 91, wherein the pomegranate extract is 5-15% (w/w). 92. The dissolvable matrix of any one of embodiments 86 to 91, wherein the pomegranate extract is 8% (w/w). 93. The dissolvable matrix of any one of embodiments 55 to 92 comprises a hygroscopicity modifier. 94. The dissolvable matrix of embodiment 93, wherein the hygroscopicity modifier is a medium chain triglyceride (MCT) oil powder. 95. The dissolvable matrix of any one of embodiments 55 to 94, wherein the excipient comprises a pore-creating excipient. 96. The dissolvable matrix of embodiment 95, wherein the excipient is a pore-creating excipient is a microcrystalline cellulose. 97. The dissolvable matrix of any one of embodiments 55 to 96, wherein the excipient comprises quillaja extract and/or powdered cellulose. 98. A dissolvable matrix comprising: at least 30% (w/w) of one or more active agents, wherein one or more of the active agents is a sleep enhancer; and at least one excipient, wherein the matrix is configured to dissolve in an aqueous solution. 99. The dissolvable matrix of embodiment 98, wherein the sleep enhancer is selected from chamomile extract, L-theanine, or melatonin. 100. The dissolvable matrix of embodiment 98 or embodiment 99, wherein the matrix comprises chamomile extract, L-theanine, and melatonin. 101. The dissolvable matrix of any one of embodiments 98 to 100, wherein the excipient is selected from pullulan, oat fiber, or CMC gum. 102. The dissolvable matrix of any one of embodiments 98 to 101, wherein the excipient comprises quillaja extract and/or powdered cellulose. 103. A dissolvable matrix comprising: at least 30% (w/w) of one or more active agents, wherein one or more of the active agents is an immunity enhancer; and at least one excipient, wherein the matrix is configured to dissolve in an aqueous solution. 104. The dissolvable matrix of embodiment 103, wherein the active agent is selected from a phytosome, a vitamin, or mineral. 105. The dissolvable matrix of embodiment 104, wherein the matrix comprises a phytosome, a vitamin, and a mineral. 106. The dissolvable matrix of embodiment 104 or 105, wherein the lecithinized product is quercetin lecithin complex. 107. The dissolvable matrix of any one of embodiments 103 to 106, wherein the vitamin is vitamin D3 or ascorbic acid. 108. The dissolvable matrix of any one of embodiments 103 to 107, wherein the mineral is a zinc salt. 109. The dissolvable matrix of embodiment 108, wherein the zinc salt is zinc picolinate. 110. The dissolvable matrix any one of embodiments 103 to 109, wherein the excipient is selected from pullulan, tapioca starch, or calcium carbonate. 111. The dissolvable matrix any one of embodiments 103 to 110, wherein the excipient comprises quillaja extract and/or powdered cellulose. 112. The dissolvable matrix of any one of embodiments 103 to 111, wherein the excipient comprises a pore-creating excipient. 113. The dissolvable matrix of embodiment 112, wherein the pore-creating excipient is Tapioca starch. 114. A dissolvable matrix comprising: at least 30% (w/w) of one or more active agents, wherein one or more of the active agents is a performance enhancer; and at least one excipient, wherein the matrix is configured to dissolve in an aqueous solution. 115. The dissolvable matrix of embodiment 114, wherein the active agent is selected from a mango leaf extract, methylcobalamin, L-methyltetrahydrofolate Ca, and pyridoxal-5-phosphate. 116. The dissolvable matrix of embodiment 115, wherein the active agent is two or more of mango leaf extract, methylcobalamin, L-methyltetrahydrofolate Ca, and pyridoxal-5-phosphate. 117. The dissolvable matrix of embodiment 116, wherein the active agent is three or more of mango leaf extract, methylcobalamin, L-methyltetrahydrofolate Ca, and pyridoxal-5-phosphate. 118. The dissolvable matrix of embodiment 116, wherein the active agent comprises mango leaf extract, methylcobalamin, L-methyltetrahydrofolate Ca, and pyridoxal-5-phosphate. 119. The dissolvable matrix of any one of embodiments 114 to 118, wherein the excipient comprises quillaja extract and/or powdered cellulose. 120. The dissolvable matrix any one of embodiments 114 to 119, wherein the excipient comprises a pore-creating excipient. 121. The dissolvable matrix of embodiment 120, wherein the pore-creating excipient is microcrystalline cellulose. 122. The dissolvable matrix of any one of embodiments 114 to 121, wherein the excipient comprises a hygroscopicity modifier. 123. The dissolvable matrix of embodiments 122, wherein the hygroscopicity modifier is a medium chain triglyceride (MCT) oil powder. 124. A method for producing a dissolvable matrix comprising: a. mixing one or more active agents and at least one excipient in a solvent to form a mixture; b. printing the mixture; and c. curing the mixture until it comprises no more than 4% water (w/w) to form a dissolvable matrix, wherein the ratio of the one or more active agents to the at least one excipient is at least 50% (w/w). 125. The method of embodiment 124, where the solvent is water or ethanol. 126. The method of embodiment 124 or 125, where the solvent is at least 30% (w/w) prior to curing. 127. The method of embodiment 124 or 125, where the solvent is 30-60% (w/w) prior to curing.

### Definitions

As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “dissolving,” “dissolve,” “dissolvable,” etc. encompass disintegration of a composition (e.g., dissolvable matrix or dissolvable sheet) described herein. In some instances, dissolving refers to a state wherein at least 80%, 85%, 90%, 95%, 97%, or at least 99% of the composition disintegrates in liquid medium. In some instances, after disintegration, the dissolvable matrix has particle sizes (as measured by volume) no greater than 0.00001%, 0.0001%, 0.001%, 0.01%, or 0.1% of the original volume of the dissolvable matrix. In some instances, additional methods are used to describe dissolution including break timing (time required to observe fracturing of a dissolvable matrix as it rests on the surface of a liquid with no force applied other than from the liquid itself). In some instances, fracturing is observed with the naked eye (without an magnification aid).

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Preparation of an Illustrative Dissolvable Matrix 1 for Prebiotic Supplement

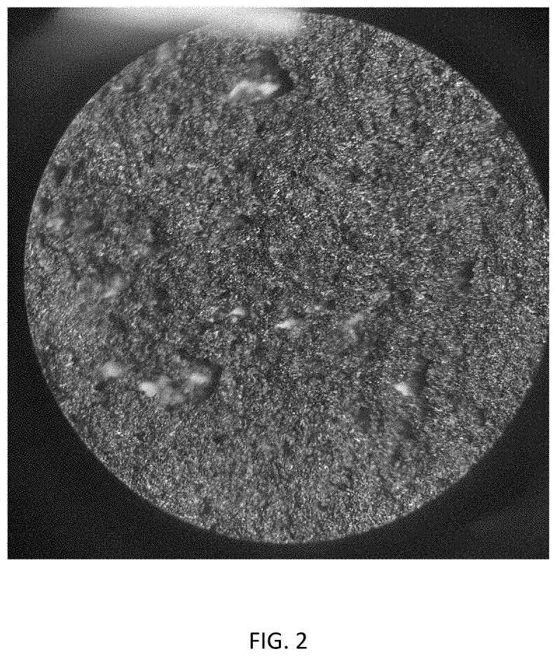

A dissolvable matrix for digestive health was prepared. Dry materials comprising a mixture of a bacteriophage component (10-20 g), blueberry powder (100-200 g), green tea extract (100-200 g), pomegranate powder (20-100 g), pullulan (10-50 g), xylitol (30-75 g), refined glycerin (25-70 g), and acerola powder (8-20 g) were blended into a premix and place into 520 g of purified water at 60° C. Material was then mixed at variable RPMs for 15 min to achieve the proper rheology and viscosity range (5000-13000 cP) for printing. After target viscosity was achieved the material was placed or pumped to the printer and stencil. The stencil was set to specific gauge of 20. Squeegee durometer, edge shape, and blade orientation also guided weight of deposition. After printing in the shape of a disc, the material was cured in a convection oven at 70° C. until the resulting matrix contained less than 4% moisture. Each disc had a dry weight of approximately 590 g. An exemplary disc after curing is shown in FIG. 2.

### Example 2: Preparation of an Illustrative Dissolvable Matrix 1 Comprising B Vitamins

The general procedure of Example 1 was followed, with modification: 350 mg of purified water, 20 mg of pullulan, 55 mg of xylitol, 80 mg of powdered cellulose, 50 mg of riboflavin-5-phosphate, 1.64 mg of methylcobalamin, 1.4 mg of L-methyltetrahydrofolate (L-MTHF) calcium salt, 15.38 mg of pyridoxal-5-phosphate, 25 mg of thiamine HCl, 5 mg of quillaja extract powder, 20 mg of refined glycerine, 40 mg of microcrystalline cellulose, 17.5 mg of Stevia extract (leaf) (Stevia rebaudiana), 6 mg of citric acid, 20 mg of flavor, and 15 mg of medium-chain triglyceride (MCT) oil powder were mixed to form a liquid composition. For scale-up production, 17.50 g, 1.00 g, 2.75 g, 4.00 g, 2.50 g, 0.08 g, 0.07 g, 0.77 g, 1.25 g, 0.25 g, 1.00 g, 2.00 g, 0.88 g, 0.30 g, 1.00 g, 0.75 g, respectively, of the ingredients were combined. The liquid compositions were mixed until an initial viscosity of 5000-12500 (cP) was achieved. The liquid composition was printed into a 20-gauge stencil. After curing at 70° C. and until the matrices comprised less than 4% moisture, the final solid, dissolvable matrix comprised 5.68% pullulan, 15.63% xylitol, 22.73% powdered cellulose, 14.21% riboflavin-5-phosphate, 0.47% methylcobalamin, 0.40% L-MTHF calcium salt, 4.37% pyridoxal-5-phosphate, 7.10% thiamine HCl, 1.42% quillaja extract powder, 5.68% refined glycerine, 11.37% microcrystalline cellulose, 4.97% Stevia extract (leaf) (Stevia rebaudiana), 1.70% citric acid, and 4.26% MCT oil powder (w/w) relative to the entire dissolvable matrix.

### Example 3: Preparation of an Illustrative Dissolvable Matrix 1a for Prebiotic Supplement

The general procedure of Example 1 was followed, with modification: initial mixing also included components of quillaja extract powder (5-20 g), microcrystalline cellulose (30-50 g), Stevia leaf (10-20 g), flavoring (10-40 g), and MCT oil powder (10-30 g). Each disc had a dry weight of approximately 630 g.

### Example 4: Preparation of an Illustrative Dissolvable Matrix 1a for Prebiotic Supplement

The general procedure of Example 1 was followed, with modification: 525.0 mg of purified water, 24.3 mg of pullulan, 89.1 mg of xylitol, 120.0 mg of powdered cellulose, 100.0 mg of blueberry powder, 100.0 mg of green tea decaffeinated extract (leaf) (Camellia stinensis), 50.0 mg of pomegranate polyphenol powder, 10.0 mg of quillaja extract powder, 30.0 mg of refined glycerin, 18.5 mg of a bacteriophage component, 10.4 mg of acerola powder, 47.5 mg of microcrystalline cellulose (MCC), 17.5 mg of Stevia extract (leaf) (Stevia rebaudiana), 30.0 mg of pomberry flavor, and 20.0 mg of MCT oil powder were mixed to form a liquid composition. For scale-up production, 20790.0 g, 962.28 g, 3527.57 g, 4752.0 g, 3960.0 g, 3960.0 g, 1980.0 g, 396.0 g, 1188.0 g, 731.10 g, 411.01 g, 1881.0 g, 693.0 g, 1188.0 g, and 792.0 g, respectively, of the ingredients were combined. The liquid compositions were mixed until an initial viscosity of 5000-13000 (cP) was achieved. The liquid composition was printed into a 20-gauge stencil. After curing at 70° C. and until the matrices comprised less than 4% moisture, the final solid, dissolvable matrix comprised 18.83% powdered cellulose, 15.69% blueberry powder, 15.69% green tea decaffeinated extract (leaf) (Camellia stinensis), 7.85% pomegranate polyphenol powder, 1.57% quillaja extract powder, 4.71% refined glycerin, 2.90% bacteriophage component, 1.63% acerola powder, 7.45% microcrystalline cellulose (MCC), 2.75% Stevia extract (leaf) (Stevia rebaudiana), and 3.14% MCT oil powder (w/w) relative to the entire dissolvable matrix.

### Example 5: Preparation of Two Illustrative Dissolvable Matrices 1a for Performance Enhancement

The general procedure of Example 1 was followed for the first illustrative matrix, with modification: 525.0 mg of purified water, 24.300 mg of pullulan, 89.080 mg of xylitol, 120.0 mg of powdered cellulose, 300.0 mg of mango leaf extract, 1.64 mg of methylcobalamin, 1.4 mg of L-methyltetrahydrofolate Ca, 5.38 mg of pyridoxal-5-phosphate, 10.0 mg of quillaja extract powder, 30.0 mg of refined glycerin, 47.500 mg of microcrystalline cellulose (MCC), 17.500 mg of Stevia extract (leaf) (Stevia rebaudiana), 10.0 mg of citric acid, 30.0 mg of mango flavor, and 20.0 mg of MCT oil powder were mixed to form a liquid composition. For scale-up production, 26.25 g, 1.22 g, 4.45 g, 6.0 g, 15.0 g, 0.08 g, 0.07 g, 0.27 g, 0.50 g, 1.50 g, 2.38 g, 0.88 g, 0.50 g, 1.50 g, and 1.0 g, respectively, of the ingredients were combined. The liquid compositions were mixed until an initial viscosity of 8775 (cP) and/or 11.7% torque was achieved. The liquid composition was printed into a 16-gauge stencil. After curing at 90° C. and until the matrices comprised less than 4% moisture, the final solid, dissolvable matrix comprised 3.59% pullulan, 13.16% xylitol, 17.73% powdered cellulose, 44.33% mango leaf extract, 0.24% methylcobalamin, 0.21% L-methyltetrahydrofolate Ca, 0.79% pyridoxal-5-phosphate, 1.48% quillaja extract powder, 4.43% refined glycerin, 7.02% microcrystalline cellulose (MCC), 2.59% Stevia extract (leaf) (Stevia rebaudiana), 1.48% citric acid, 2.96% MCT oil powder (w/w) relative to the entire dissolvable matrix.

The general procedure of Example 1 was followed for the second illustrative matrix, with modification: 525.0 mg of purified water, 29.0 mg of pullulan, 89.080 mg of xylitol, 120.0 mg of powdered cellulose, 300.0 mg of mango leaf extract, 1.64 mg of methylcobalamin, 1.4 mg of L-methyltetrahydrofolate Ca, 5.38 mg of pyridoxal-5-phosphate, 10.0 mg of quillaja extract powder, 40.0 mg of refined glycerin, 47.500 mg of microcrystalline cellulose, 17.500 mg of Stevia extract (leaf) (Stevia rebaudiana), 15.0 mg of citric acid, 35.0 mg of mango flavor, and 20.0 mg of MCT oil powder were mixed to form a liquid composition. For scale-up production, 26.25 g, 1.45 g, 4.45 g, 6.0 g, 15.0 g, 0.08 g, 0.07 g, 0.27 g, 0.50 g, 2.0 g, 2.38 g, 0.88 g, 0.75 g, 1.75 g, and 1.0 g, respectively, of the ingredients were combined. The liquid compositions were mixed until an initial viscosity of 8775 (cP) and/or 11.7% torque was achieved. The liquid composition was printed into a 16-gauge stencil. After curing at 90° C. and until the matrices comprised less than 4% moisture, the final solid, dissolvable matrix comprised 4.16% pullulan, 12.79% xylitol, 17.23% powdered cellulose, 43.07% mango leaf extract, 0.24% methylcobalamin, 0.20% L-methyltetrahydrofolate Ca, 0.77% pyridoxal-5-phosphate, 1.44% quillaja extract powder, 5.74% refined glycerin, 6.82% microcrystalline cellulose, 2.51% Stevia extract (leaf) (Stevia rebaudiana), 2.15% citric acid, and 2.87% MCT oil powder.

### Example 6: Preparation of an Illustrative Dissolvable Matrix 2 for Sleep Improvement

A dissolvable matrix for improved sleep was prepared. The general procedure of Example 1 was followed, with modification: the dry materials were pullulan (10-30 g), xylitol (10-30 g), powdered cellulose (70-150 g), chamomile extract (50-150 g), L-theanine (150-300 g), melatonin (0.1-5 mg), quillaja extract powder (30-50 g), refined glycerin (60-90 g), oat fiber (10-30 g), Stevia extract (5-15 g), citric acid (5-10 g), carboxymethyl cellulose (CMC) gum (5-10 g), flavoring (15-50 g), and coloring agent (10-30 g). The initial viscosity was 7000-20000 cP, and a 16-gauge stencil was used.

### Example 7: Preparation of Another Illustrative Dissolvable Matrix 2 for Sleep Improvement

The general procedure of Example 1 was followed, with modification: 400.0 mg of purified water, 23.0 mg of pullulan, 20.0 mg of xylitol, 100.0 mg of powdered cellulose, 100.0 mg of chamomile extract, 210.53 mg of L-theanine, 1.10 mg of melatonin, 40.0 mg of quillaj a extract powder, 80.0 mg of refined glycerin, 21.0 mg of oat fiber, 10.0 mg of Stevia extract (leaf) (Stevia rebaudiana), 6.0 mg of citric acid, 1.0 mg of CMC gum, 30.0 mg of blueberry flavor, 20.0 mg of blue powder were mixed to form a liquid composition. For scale-up production, 11200.0 g, 644.0 g, 560.0 g, 2800.0 g, 2800.0 g, 5894.84 g, 30.80 g, 1120.0 g, 2240.0 g, 588.0 g, 280.0 g, 168.0 g, 28.0 g, 840.0 g, and 560.0 g, respectively, of the ingredients were combined. The liquid compositions were mixed until an initial viscosity of 7000-20000 (cP) was achieved. The liquid composition was printed into a 16-gauge stencil. After curing at 80° C. and until the matrices comprised less than 4% moisture, the final solid, dissolvable matrix comprised 3.64% pullulan, 3.16% xylitol, 15.81% powdered cellulose, 15.81% chamomile extract, 33.28% L-theanine, 0.17% melatonin, 6.32% quillaja extract powder, 12.65% refined glycerin, 3.32% oat fiber, 1.58% Stevia extract (leaf) (Stevia rebaudiana), 0.95% citric acid, 0.16% CMC gum, and 3.16% blueberry flavor powder (w/w) relative to the entire dissolvable matrix.

### Example 8: Preparation of an Illustrative Dissolvable Matrix 2a for Immunity

A dissolvable matrix improved immunity was prepared. The general procedure of Example 1 was followed, with modification: the dry materials were pullulan (10-30 g), calcium carbonate (15-40 g), powdered cellulose (20-40 g), quercetin lecithin (100-400 g), vitamin D3 veg (vegetable) powder (10-30 g), ascorbic acid (40 mesh, 90-150 g), zinc picolinate (50-90 g), refined glycerin (25-75 g), tapioca starch (50-90 g), Stevia extract (15-30 g), citric acid (10-20 g), quillaja extract powder (20-40 g), flavoring (20-50 g). The amount of water used was approximately 620 g, the initial viscosity was 3000-10000 cP, and a 14-gauge stencil was used.

### Example 9: Preparation of Illustrative Dissolvable Matrix 2a for Immunity

The general procedure of Example 1 was followed, with modification: 620.0 mg of purified water, 20.0 mg of pullulan, 10.0 mg of calcium carbonate, 140.0 mg of powdered cellulose, 250.0 mg of quercetin lecithin, 30.0 mg of vitamin D3 veg powder, 115.0 mg of ascorbic acid 40 mesh, 75.0 mg of zinc picolinate, 60.0 mg of refined glycerin, 10.0 mg of tapioca starch, 34.0 mg of Stevia extract (leaf) (Stevia rebaudiana), 19.0 mg of citric acid, 35.0 mg of quillaja extract powder, and 40.0 mg of natural Meyer lemon flavor were mixed to form a liquid composition. For scale-up production, 18600.0 g, 600.0 g, 300.0 g, 4200.0 g, 7500.0 g, 900.0 g, 3450.0 g, 2250.0 g, 1800.0 g, 300.0 g, 1020.0 g, 570.0 g, 1050.0 g, and 1200.0 g, respectively, of the ingredients were combined. The liquid compositions were mixed until an initial viscosity of 40000-10000 (cP) was achieved. The liquid composition was printed into a 16-gauge stencil. After curing at 80° C. and until the matrices comprised less than 4% moisture, the final solid, dissolvable matrix comprised 2.39% pullulan, 1.19% calcium carbonate, 16.71% powdered cellulose, 29.83% quercetin lecithin, 3.58% vitamin D3 veg powder, 13.72% ascorbic acid 40 mesh, 8.95% zinc picolinate, 7.16% refined glycerin, 1.19% tapioca starch, 4.06% Stevia extract (leaf) (Stevia rebaudiana), 2.27% citric acid, 4.18% quillaja extract powder, and 4.77% natural Meyer lemon flavor (w/w) relative to the entire dissolvable matrix.

### Example 10: Pore Analysis Via Disk Scanning Electron Microscopy

Dissolvable matrices were generated in the shape of 2 inch diameter discs using the general methods of Examples 1-9 and analyzed using SEM.

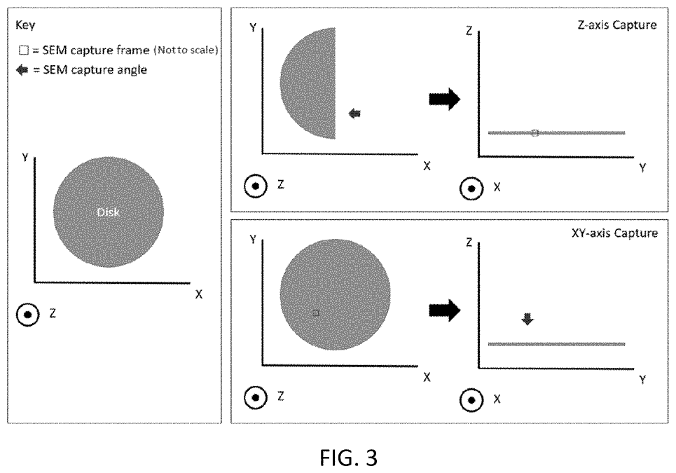

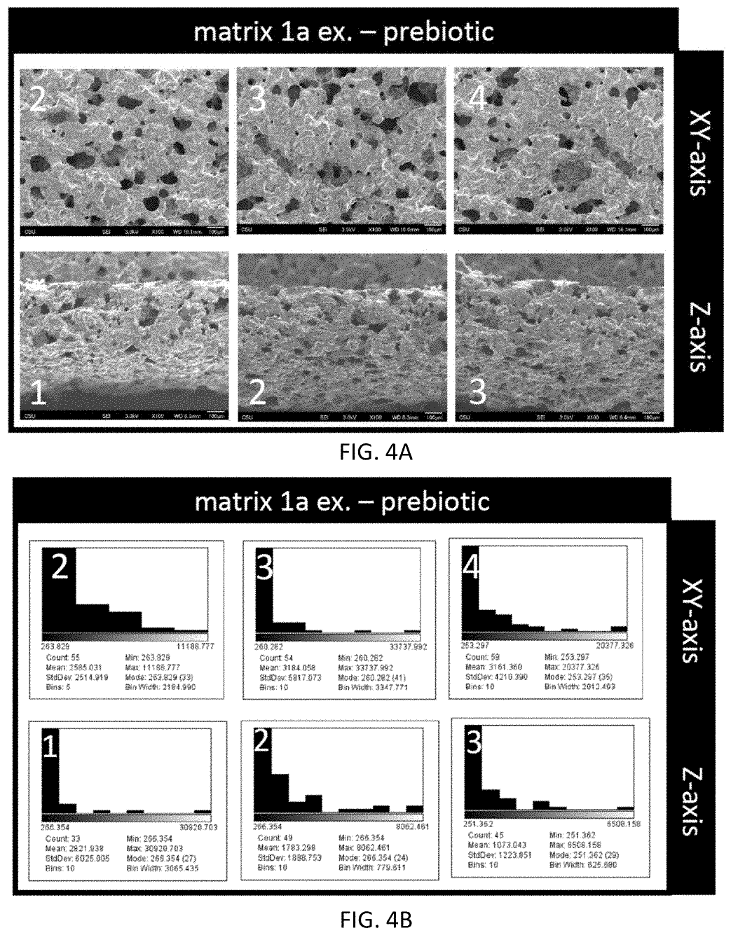

SEM Analysis of Physical Disk. In order to analyze the disks in three dimensions, two sets of SEM images were obtained (FIG. 3). The first image acquired is of the XY-axis and images the surface of the disk via mounting to the SEM stud. The surface of the disk which was not contacting a solid support (i.e., top side) during curing was used for imaging the XY axis. A second set of images were obtained from the Z-axis, wherein the disk was sectioned and mounted. The sectioning of the disk was done at room temperature with a standard single edge razor blade. In order to mount the Z-axis disk, three sections were cut and the middle section was turned 90° and sandwiched between the other disks. This sandwich structure was then mounted onto the SEM stub. Once each disk had representative XY-axis and Z-axis stubs, the samples were imaged in a scanning electron microscope at 3.0 kV. During the imaging process, different parts of the XY-axis and Z-axis were imaged multiple times in order to determine intra-disk variations. This process produced a set of images for image analysis. Representative images are shown in FIGS. 4A, 5A, and 6A.

ImageJAnalysis of Disk SEM Images. In the image analysis phase, three XY-axis images and three Z-axis images (for a total of 6 images) were chosen from the set of SEM images. In some cases, only two images were representative of the actual disk and were used in the image analysis process. Once the six images were selected, the following process was used to analyze the pores in the disk superstructure (using ImageJ software). The Image J workflow was as follows: 1) image was opened in ImageJ, 2) image scale was set, 3) image was duplicated, 4) image was set to 8-bit grayscale type, 5) color threshold was set to represent pores in original image, 6) image was touched up based on features in original image, 7) particles were analyzed by sorting by minimum pore size (no max pore size is needed), and 8) data was logged.

ImageJAnalysis of Disk SEM Images. In the next phase of the analytical process from the logged data, the metrics obtained were: 1) void volume (% of total focal area), 2) mean more size (μm²), 3) the standard deviation of pore size (μm²), 4) median pores area (μm²), 5) pores per area (pores/mm²), and 6) the distribution of pores. Each image was analyzed to create a pore distribution that was characterized by the mean, median, and standard deviation (FIGS. 4B, 5B, and 6B). Pores were counted based on adherence to actual features within the original image. In some instances this was used to calculate the number of pores per square millimeter. Distributions were binned by average pore size. The number of bins were determined by the number of total observations.

### Example 11: Dissolvable Matrices

Dissolvable matrices (disk/sheet shaped) were prepared using the general methods of Examples 1-9. The general procedure for producing the disks included: ingredients were mixed together according to Table 3 below until a cP within the range of 5000-12000 cP was achieved. For optimal mixing the mixing implement tip speed was 1-1500 FPM. Once the optimal cP within the range was reached, the composition was aliquoted onto a 2D screen printer with specified squeegee pressure of 1-100 kgf applied to stencil and hydrous material, which allows the disc to meet the specified unit dose weight dependent on the example. Stencil thickness was from 0.700 mm-4.0 mm. Once printed, hydrous disks on the substrate were set to cure. Hydrous disk heights mirrored stencil parameters at 0.700 mm-4.0 mm. Curing temp was set to 45-110 degrees C. depending on the example and cured for 15-180 minutes. Once cooled, disks were removed from the substrate and set to package. Disks produced in this manner were 25.4-53.34 mm in diameter and 0.3-3.0 mm thick with moisture limits set to less than 4%.

Ingredients used to produce the dissolvable matrices are shown in Table 3.

TABLE 3

| Disk ID | | 001 | 002 | 003 | 004 |
|---|---|---|---|---|---|
| API/Active agent mixture type/Use | | Prebiotic | Sleep | Immunity | Performance |
| Matrix | | 1a | 2 | 2a | 2 |
| Ingredient | Type | % Anhydrous Composition (maximum, w/w) | | | |
| blueberry powder | Active Agent | 15.47% | — | — | — |
| green tea decaffeinated extract (leaf) | Active Agent | 15.47% | — | — | — |
| (camellia stinensis) | | | | | |
| pomegranate polyphenol powder | Active Agent | 7.74% | — | — | — |
| Preforpro (a prebiotic bacteriophage cocktail) | Active Agent | 2.86% | — | — | — |
| Chamomile Extract | Active Agent | — | 15.30% | — | — |
| L-Theanine | Active Agent | — | 32.21% | — | — |
| Melatonin | Active Agent | — | 0.17% | — | — |
| Quercetin Phytosome Maltodextrin Free | Active Agent | — | — | 28.52% | — |
| Vitamin D3 Veg Powder | Active Agent | — | — | 3.42% | — |
| Ascorbic Acid 40 Mesh | Active Agent | — | — | 13.12% | — |
| Zinc Picolinate | Active Agent | — | — | 8.56% | — |
| mango extract | Active Agent | — | — | — | 33.33% |
| methyl cobalamin | Active Agent | — | — | — | 0.15% |
| L-5-methyltetrahydrofolate | Active Agent | — | — | — | 0.09% |
| pyridoxal-5-phosphate | Active Agent | — | — | — | 0.60% |
| riboflavin-5-phosphate sodium | Active Agent | — | — | — | 0.34% |
| quillaja extract powder | Blowing Agent | 1.55% | 6.12% | 4.45% | 4.44% |
| (no less than 20% saponins) | | | | | |
| pullulan hppe | Scaffolding Agent | 3.76% | 4.44% | — | — |
| powdered cellulose | Scaffolding Agent | 18.57% | 15.30% | 15.97% | 13.33% |
| Microcrystalline Cellulose (Heweten 101) | Scaffolding Agent | 7.35% | — | — | 2.50% |
| Oat Fiber | Scaffolding Agent | — | 3.21% | — | 16.66% |
| Citric Acid | Scaffolding Agent | — | 0.92% | 2.28% | |
| Pullulan | Scaffolding Agent | — | — | 3.99% | 2.22% |
| Calcium Carbonate | Scaffolding Agent | — | — | 0.80% | — |
| Tapioca Starch | Scaffolding Agent | — | — | 1.14% | — |
| Potassium Bicarbonate | Scaffolding Agent | — | — | — | 1.11% |
| xylitol | Humectant | 13.78% | 3.06% | 4.56% | 6.67% |
| refined glycerine | Humectant | 4.64% | 12.24% | — | 5.55% |
| acerola powder | Humectant | 1.61% | — | — | — |
| Glycerin (Vegetable Source) | Humectant | — | — | 4.56% | — |
| medium-chain triglyceride (MCT) Oil | Hygroscopicity | 3.09% | — | — | 2.78% |
| Powder | Modifier | — | — | — | — |
| carboxymethyl cellulose (CMC) Gum | Hygroscopicity | — | 1.07% | — | — |
| | Modifier | — | — | — | — |
| Stevia Extract (Leaf) (Stevia rebaudiana) | Flavor | 2.71% | 1.53% | 3.99% | 1.67% |
| PomBerry Flavor 589897RO | Flavor | 1.39% | — | — | — |
| Blueberry Flavor 823166RO | Flavor | — | 1.38% | — | — |
| natural flavor blend modifier type 474404RO | Flavor | — | — | 2.05% | — |
| Natural Meyer Lemon Flavor 823857RO | Flavor | — | — | 2.57% | — |
| Natural Orange Flavor WONF 282942RO | Flavor | — | — | — | 2.22% |
| natural tropical punch flavor 465655RO | Flavor | — | — | — | 1.33% |
| natural mango flavor 185850RO | Flavor | — | — | — | 3.33% |
| Blue Microfine SE Powder | Colorant | — | 3.06% | — | — |
| Yellow Microfine | Colorant | — | — | — | 1.67% |

### Example 12: Properties of Dissolvable Matrices

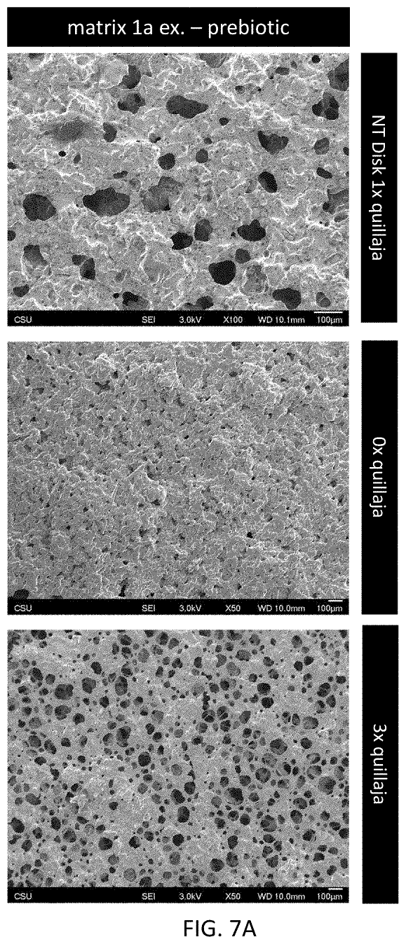

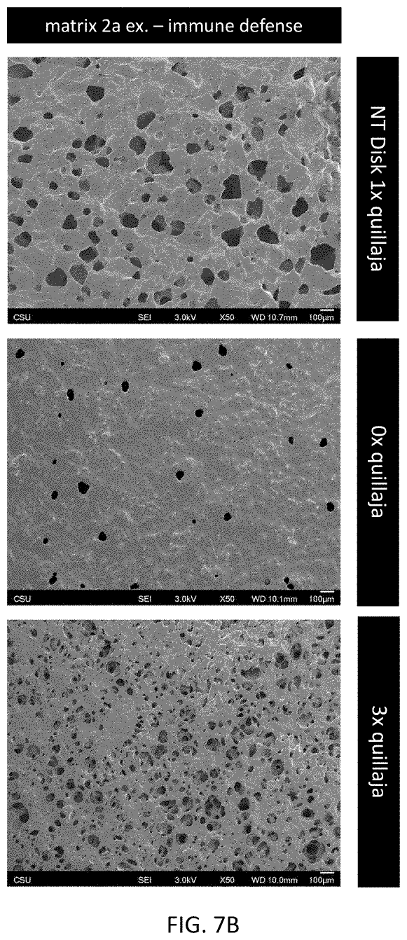

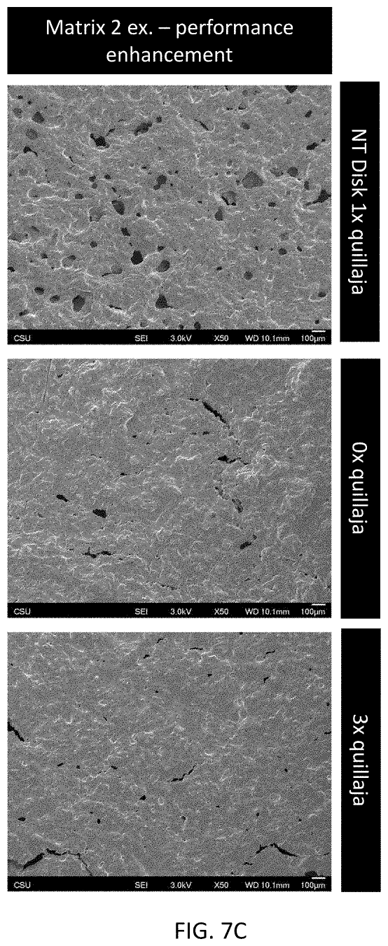

Dissolvable matrices of Example 11 were subjected to SEM pore analysis using the general methods of Example 10. Comparator disks containing either no quillaj a or three fold excess quillaja were also evaluated. Higher amounts of quillaja increased porosity of the resulting dissolvable matrices (FIG. 7A-7C, bottom images). A summary of pore properties for disks of this and previous examples is described in Table 4.

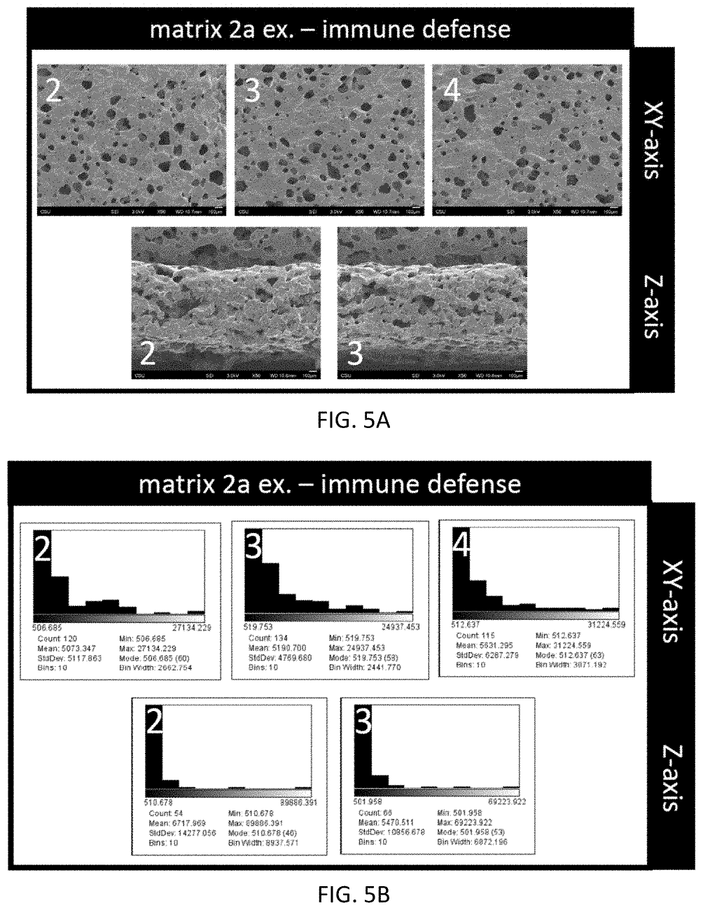

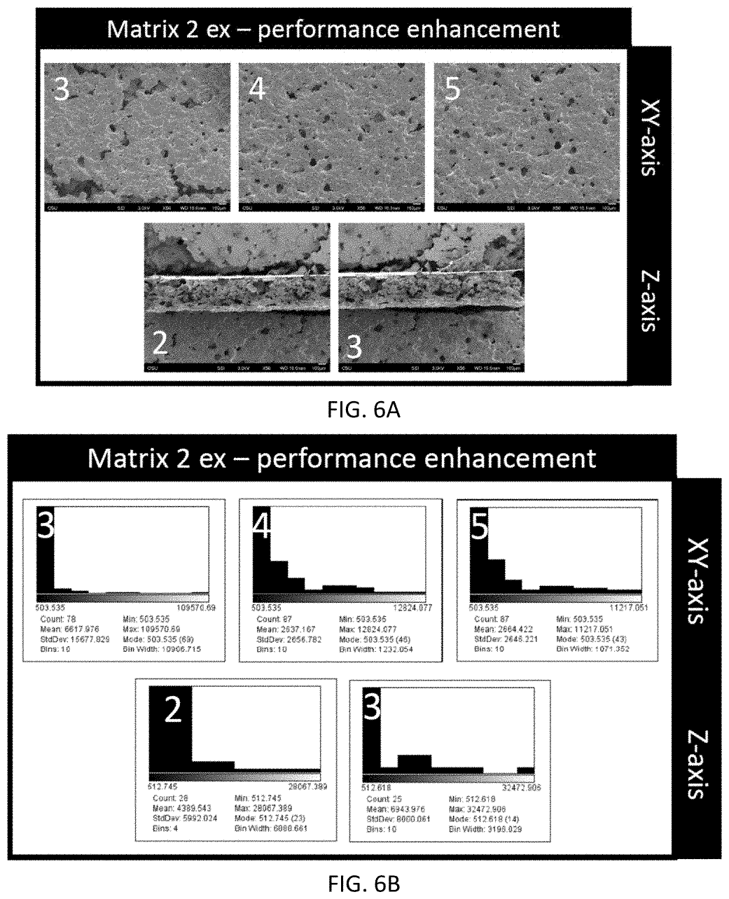

TABLE 4

| | | Pore distribution | | | | |
|---|---|---|---|---|---|---|
| | | | mean | | | |
| | | void volume | pore | Pore area | Median | Pore Count |
| Disk | SEM Images/ | (% of total | area* | standard | pore area | Pores per area |
| ID | Data | focal area) | (μm²) | deviation (μm²) | (μm²) | (pores/mm²) |
| 001 | FIGS. 4A-4B; | 15.56 | 2976.82 | 4180.79 | 1255.35 | 52.21 |
| | FIG. 7A, top | | | | | |
| | image | | | | | |
| 001^{a} | FIG. 7A, | 3.88 | 1243.38 | 1910.21 | 824.40 | 31.71 |
| | middle image | | | | | |
| 001^{b} | FIG. 7A, | 26.30 | 2864.68 | 4401.08 | 1908.12 | 91.87 |
| | bottom image | | | | | |
| 003 | FIGS. 5A-5B; | 14.86 | 5298.45 | 5391.61 | 3331.27 | 28.10 |
| | FIG. 7B, top | | | | | |
| | image | | | | | |
| 003^{a} | FIG. 7B, | 1.15 | 2216.07 | 1375.13 | 2044.71 | 5.16 |
| | middle image | | | | | |
| 003^{b} | FIG. 7B, | 17.30 | 1885.47 | 2073.45 | 1226.07 | 91.32 |
| | bottom image | | | | | |
| 004 | FIGS. 6A-6B; | 7.45 | 3973.19 | 6993.61 | 1612.98 | 19.21 |
| | FIG. 7C, top | | | | | |
| | image | | | | | |
| 004^{a} | FIG. 7C, | 1.31 | 2379.39 | 2947.90 | 1005.47 | 5.31 |
| | middle image | | | | | |
| 004^{b} | FIG. 7C, | 3.10 | 2065.36 | 2536.49 | 1043.73 | 2.26 |
| | bottom image | | | | | |

*cross-sectional area;
^{a}contains no quillaja - middle image in FIGS. 7A-7C;
^{b}contains threefold excess quillaja - bottom image in FIGS. 7A-7C.

Dissolvable matrices of Example 11 were also analyzed for performance. Hygroscopicity was measured at 20 degrees C. at 1 atm under 45% relative humidity. Z-axis were measured using a 0-150 mm digital caliper. A summary of hygroscopicity for the disks of this and previous examples is described in Table 5.

TABLE 5

| | | | | | | | Rate | |
|---|---|---|---|---|---|---|---|---|
| | | Cumulative time (min)/Water activity, % moisture content (db) | | | | | Water | % moisture |
| Disk | Z-axis | 5.0 | 30.0 | 60.0 | 120.0 | 240.0 | activity | content |
| ID | (mm) | min | min | min | min | min | (per min) | (%/min) |
| 001 | 0.765 | 0.2400, | 0.2861, | 0.3326, | 0.4062, | 0.4434, | 0.00083837 | 0.0193 |
| | | 2.01% | 2.61% | 3.60% | 5.51% | 6.45% | | |
| 001^{a} | 0.850 | 0.2618, | 0.3620, | 0.4219, | 0.4446, | 0.4491, | 0.00063023 | 0.0140 |
| | | 2.39% | 4.48% | 5.98% | 6.45% | 6.50% | | |
| 001^{b} | 0.600 | 0.1824, | 0.2350, | 0.2988, | 0.4083, | 0.4237, | 0.00101617 | 0.0206 |
| | | 1.40% | 1.92% | 2.85% | 5.51% | 5.98% | | |
| 003 | 1.2 | 0.2200, | 0.2398, | 0.2647, | 0.3136, | 0.4008, | 0.000771 | 0.0146 |
| | | 0.75% | 1.11% | 1.51% | 2.33% | 4.20% | | |
| 003^{a} | 1.330 | 0.2245, | 0.3407, | 0.4218, | 0.4362, | 0.4398, | 0.000712 | 0.0143 |
| | | 0.87% | 2.94% | 4.62% | 4.84% | 5.10% | | |
| 003^{b} | 0.90 | 0.1838, | 0.2507, | 0.2717, | 0.3519, | 0.3553, | 0.000673 | 0.0123 |
| | | 0.20% | 1.25% | 1.58% | 3.17% | 3.23% | | |
| 004 | 0.78 | 0.1853, | 0.2300, | 0.2769, | 0.3625, | 0.4375, | 0.001058 | 0.0191 |
| | | 0.78% | 1.52% | 2.20% | 3.76% | 5.30% | | |
| 004^{a} | 0.81 | 0.1838, | 0.2691, | 0.3725, | 0.4376, | 0.4382, | 0.000974 | 0.0181 |
| | | 0.75% | 2.10% | 4.00% | 5.30% | 5.35% | | |
| 004^{b} | 0.58 | 0.1836, | 0.2592, | 0.3701, | 0.4254, | 0.4363, | 0.000976 | 0.0178 |
| | | 0.74% | 1.93% | 3.96% | 5.02% | 5.27% | | |

^{a}contains no quillaja;
^{b}contains three fold excess quillaja.

To measure dissolution properties, disks were subjected to a break test wherein the disk was placed in 200 mL of tap water at 22.2 degrees C. The break time was defined as the time required to observe fracturing of the disk as it rests on the surface of water with no force applied other than from the water itself. Dissolution rates were also measured using mechanical stirring (200 mL tap water at 22.2 degrees C., stirred with a stainless steel spatula), which was applied after fracturing was observed. Break times and dissolution rates are shown in Table 6.

TABLE 6

| Disk | Disk Break | Mechanical Force Applied (stirring) |
|---|---|---|
| ID | (minutes:seconds) | (minutes:seconds, cumulative) |
| 001 | 0:25 | 1:45 |
| 001^{a} | 2:58 | 14:03 |
| 001^{b} | 0:04 | 0:15 |
| 003 | 0:15 | 2:00 |
| 003^{a} | N/A* | 45:00 |
| 003^{b} | 0:07 | 0:35 |
| 004 | 0:45 | 2:00 |
| 004^{a} | 5:20 | 10:00 |
| 004^{b} | 0:35 | 1:45 |

^{a}contains no quillaja;
^{b}contains three fold excess quillaja;
*no break, disk sank to bottom.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-207. (canceled)

208. A pore-containing dissolvable sheet for delivery of one or more active agents into an aqueous medium, the dissolvable sheet comprising:
(a) one or more scaffolding agents;
(b) one or more blowing agents; and
(c) one or more active agents;
wherein the sheet, when exposed to an environment at 20 degrees Celsius, 1 atm of pressure, and 45% humidity, absorbs less than 0.003% moisture (w/w) per minute over 235 minutes beginning 5 minutes after exposure to the environment and ending 240 minutes after exposure to the environment; and
wherein the sheet, when placed on a surface of water at a water temperature of 22.2 degrees Celsius, fractures within 2 minutes without mechanical stirring.

209. The pore-containing dissolvable sheet of claim 208, wherein a surface of the dissolvable sheet comprises a plurality of pores having a longest cross-sectional length between 10 and 200 microns, wherein the plurality of pores are present on the surface of the dissolvable sheet at a density of between 10 and 100 pores per square mm, wherein a surface of the dissolvable sheet comprises a plurality of pores having an average cross-sectional area of 1000 to 4000 square microns, wherein the average cross-sectional area has a standard deviation of deviation less than 7000 square microns.

210. The pore-containing dissolvable sheet of claim 208, wherein the pore-containing dissolvable sheet has a void volume of 3-30%.

211. A pore-containing dissolvable sheet for delivery of one or more active agents into an aqueous medium, the dissolvable sheet comprising:
(a) one or more scaffolding agents;
(b) one or more blowing agents; and
(c) one or more active agents;
wherein a surface of the dissolvable sheet comprises a plurality of pores having a longest cross-sectional length between 10 and 200 microns, wherein the plurality of pores are present on the surface of the dissolvable sheet at a density of between 10 and 100 pores per square mm.

212. The pore-containing dissolvable sheet of claim 211, wherein a surface of the dissolvable sheet comprises a plurality of pores having an average cross-sectional area of 500 to 2000 square microns, wherein the average cross-sectional area has a standard deviation of deviation less than 7000 square microns.

213. The pore-containing dissolvable sheet of claim 211, wherein the pore-containing dissolvable sheet has a void volume of 3-30%.

214. The pore-containing dissolvable sheet of claim 211, wherein the sheet, when exposed to an environment at 20 degrees Celsius, 1 atm of pressure, and 45% relative humidity, absorbs less than 0.003% moisture (w/w) per minute over 235 minutes beginning 5 minutes after exposure to the environment and ending 240 minutes after exposure to the environment.

215. The pore-containing dissolvable sheet of claim 211, wherein the sheet, when mechanically stirred after initial fracture, disintegrates in water within 3 minutes.

216. The pore-containing dissolvable sheet of claim 211, wherein the sheet, when placed on a surface of water at a water temperature of 22.2 degrees Celsius, fractures within 2 minutes without mechanical stirring.

217. The pore-containing dissolvable sheet of claim 211, wherein the one or more scaffolding agents is or comprises powdered cellulose, microcrystalline cellulose, saponins, or quillaj a extract.

218. The pore-containing dissolvable sheet of claim 211, wherein the matrix further comprises an acid/base pair that, when combined, results in the evolution of a gas.

219. The pore-containing dissolvable sheet of claim 218, wherein the acid/base pair comprises citric acid, carbonate, calcium bicarbonate, or calcium citrate.

220. The pore-containing dissolvable sheet of claim 211, wherein the sheet has an average thickness of between 50 and 2000 microns, wherein the sheet has a total surface area of from 0.5 square inches and 20 square inches when the sheet is treated is having a flat external surface for the purpose determining surface area.

221. The pore-containing dissolvable sheet of claim 211, wherein the active agent is or comprises a pharmaceutical composition or a nutraceutical composition.

222. The pore-containing dissolvable sheet of claim 211, wherein the active agent is or comprises a plant extract, animal extract, fungal extract, a prebiotic, or a sleep enhancer.

223. The pore-containing dissolvable sheet of claim 211, wherein the active agent is or comprises blueberry powder, green tea decaffeinated extract (leaf), pomegranate polyphenol powder, Preforpro, Chamomile Extract, L-Theanine, Melatonin, Quercetin phytosome, Vitamin D3, Ascorbic Acid, Zinc Picolinate, mango extract, methyl cobalamin, 1-5-methyltetrahydrofolate, pyridoxal-5-phosphate, or riboflavin-5-phosphate sodium.

224. The pore-containing dissolvable sheet of claim 211, wherein the dissolvable sheet comprises at least one hygroscopicity modifier, wherein the at least one hygroscopicity modifier is or comprises medium-chain triglyceride (MCT) oil powder or carboxymethyl cellulose (CMC) gum.

225. The pore-containing dissolvable sheet of claim 211, wherein the dissolvable sheet comprises at least one humectant, wherein the at least one humectant is or comprises potassium bicarbonate, xylitol, glycerine, or acerola powder.

226. The pore-containing dissolvable sheet of claim 211, wherein the dissolvable sheet comprises no more than 4% water (w/w).

227. The pore-containing dissolvable sheet of claim 211, wherein the dissolvable sheet comprises a water activity of no more than 0.4 after no more than two hours when exposed to 20 degrees C., 1 atm, and 45% relative humidity.

228. The pore-containing dissolvable sheet of claim 211, wherein the one or more scaffolding agents are between 0.4% and 40% of the dissolvable sheet by weight.

229. The pore-containing dissolvable sheet of claim 211, wherein the one or more active agents are between 0.05% and 70% of the dissolvable sheet by weight.

230. The pore-containing dissolvable sheet of claim 211, wherein the sheet comprises a plurality of pores having a cross-sectional area between 1000 and 10,000 square microns at a density of between 10 and 100 pores per square mm.

231. The pore-containing dissolvable sheet of claim 211, wherein the weight ratio of the one or more scaffolding agents to the one or more active agents is between 1:5 to 3:1.

232. A method for manufacturing a dissolvable sheet, the method comprising:
mixing one or more scaffolding agents, one or more blowing agents, and one or more active agents to from a mixture having a viscosity from 4,000 to 15,000 cP;
depositing the mixture onto a surface having a predefined shape;
removing water from the deposited mixture to form a solid composition having a water content of less than 4% (w/w).

233. The method of claim 232, wherein depositing the mixture comprises depositing the mixture onto a stencil, wherein the stencil has a fillable height of from 0.7 mm to 4.0 mm.

234. The method of claim 232, wherein the solid composition has a thickness of between 100 microns and 3000 microns.

235. The method of claim 232, wherein removing the water from the deposited mixture comprises heating the deposited mixture, wherein the deposited mixture is heated by exposure to a temperature of between 45 and 110 degrees Celsius for between 15 and 180 minutes.

236. The method of claim 232, wherein depositing the mixture onto the surface comprises delivering the material via a printer with a squeegee pressure of 1-100 kgf.
